SEQUENCE LISTING (248 sequences)

SEQ ID NO: 1 - DNA, 5025 bp, Artificial
  Synthetic
cgcgacctcagatcagacgtggcgacccgctgaatttaagcatattagtcagcggaggaaaagaaactaaccaggattccctcagtaacggcgagtgaacagggaagagcccagcgccgaatccccgccccgcggggcgcgggacatgtggcgtacggaagacccgctccccggcgccgctcgtggggggcccaagtccttctgatcgaggcccagcccgtggacggtgtgaggccggtagcggccggcgcgcgcccgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgggtccggccgtgtcggcggcccggcggatctttcccgccccccgttcctcccgacccctccacccgccctcccttcccccgccgcccctcctcctcctccccggagggggcgggctccggcgggtgcgggggtgggcgggcggggccgggggtggggtcggcgggggaccgtcccccggaccggcgaccggccgccgccgggcgcatttccaggcggtgcgccgcgaccggctccgggacggctgggaaggcccggcggggaaggtggctcggggggccccgtccgtccgtccgtcctcctcctcccccgtctccgccccccggccccgcgtcctccctcgggagggcgcgcgggtcggggcggcggcggcggcggcggtggcggcggcggcgggggcggcgggaccgaaaccccccccgagtgttacagcccccccggcagcagcactcgccgaatcccggggccgagggagcgagacccgtcgccgcgctctcccccctcccggcgcccacccccgcgggaatccccgcgaggggggtctcccccggcgcggcgccggcgtctcctcgtgggggggccgggccacccctcccacggcgcgaccgctctcccacccctcctccccgcgcccccgccccggcgacggggggggtgccgcgcgcgggtcggggggcggggcggactgtccccagtgcgccccgggcgggtcgcgccgtcgggcccgggggaggttctctcggggccacgcgcgcgtcccccgaagagggggacggcggagcgagcgcacggggtcggcggcgacgtcggctacccacccgacccgtcttgaaacacggaccaaggagtctaacacgtgcgcgagtcgggggctcgcacgaaagccgccgtggcgcaatgaaggtgaaggccggcgcgctcgccggccgaggtgggatcccgaggcctctccagtccgccgaggggcaccaccggcccgtctcgcccgccgcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcagacccgacgcacccccgccacgcagttttatccggtaaagcgaatgattagaggtcttggggccgaaacgatctcaacctattctcaaactttaaatgggtaagaagcccggctcgctggcgtggagccggggtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcgagagtggacgggagcggcgggggcggcggcgcgcgcgcgcgtgtggtgtgcgtcggagggcggcggcggcggcggcggcgggggtgtggggtccttcccccgcccccccccccacgcctcctcccctcctcccgcccacgccccgctccccgcccccggagccccgcggagctacgccgcgacgagtaggagggccgctgcggtgagccttgaagcctagggcgcgggcccgggtggaggccgccgcaggtgcagatcttggtggtagtagcaaatattcaaacgagaactttgaaggccgaagtggagaagggttccatgtgaacagcagttgaacatgggtcagtcggtcctgagagatgggcgagcgccgttccgaagggacgggcgatggcctccgttgccctcggccgatcgaaagggagtcgggttcagatccccgaatccggagtggcggagatgggcgccgcgaggcgtccagtgcggtaacgcgaccgatcccggagaagccggcgggagccccggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgcgcgccccccccacgcccggggcacccccctcgcggccctcccccgccccacccgcgcgcgccgctcgctccctccccaccccgcgccctctctctctctctctcccccgctccccgtcctcccccctccccgggggagcgccgcgtgggggcgcggcggggggagaagggtcggggcggcaggggccgcgcggcggccgccggggcggccggcgggggcaggtccccgcgaggggggccccggggacccggggggccggcggcggcgcggactctggacgcgagccgggcccttcccgtggatcgccccagctgcggcgggcgtcgcggccgcccccggggagcccggcggcggcgcggcgcgccccccacccccaccccacgtctcggtcgcgcgcgcgtccgctgggggcgggagcggtcgggcggcggcggtcggcgggcggcggggcggggcggttcgtccccccgccctacccccccggccccgtccgccccccgttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgcggccctgcgggccgccggtgaaataccactactctgatcgttttttcactgacccggtgaggcgggggggcgagcccgaggggctctcgcttctggcgccaagcgcccgcccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcgccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcgaacgatacggcagcgccgcggagcctcggttggcctcggatagccggtcccccgcctgtccccgccggcgggccgcccccccctccacgcgccccgccgcgggagggcgcgtgccccgccgcgcgccgggaccggggtccggtgcggagtgcccttcgtcctgggaaacggggcgcggccggaaaggcggccgccccctcgcccgtcacgcaccgcacgttcgtggggaacctggcgctaaaccattcgtagacgacctgcttctgggtcggggtttcgtacgtagcagagcagctccctcgctgcgatctattgaaagtcagccctcgacacaagggtttgtc

SEQ ID NO: 2 - DNA, 2235 bp, Artificial
  Synthetic
cgctgctcctcccgtcgccgtccgggcccgtccgtccgtccgtccgtcgtcctcctcgctnnnncggggcgccgggcccgtcctcacnggcccccgnnnnngtccnggcccgtcggggcctcgccgcgctctaccttacctacctggttgatcctgccagtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattccttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcattaacggagcccggacggcggcccgcggcggcgccgcgccgcgcttccctccgcacacccacccccccaccgcgacggcgcgtgcgggcggggccgtgcccgttcgttcgctcgctcgttcgttcgccgcccggcccggccgcgagagccgagaactcgggagggagacgggggagagagagagagagagagagagagagagagagagagagagaaagaagggcgtgt

SEQ ID NO: 3 - DNA, 156 bp, Artificial
  Synthetic
cgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcgct

SEQ ID NO: 4 - DNA, 121 bp, Artificial
  Synthetic
gtctacggccataccaccctgaacgcgcccgatctcgtctgatctcggaagctaagcagggtcgggcctggttagtacttggatgggagaccgcctgggaataccgggtgctgtaggcttt

SEQ ID NO: 5 - DNA, 1558 bp, Artificial
  Synthetic
gctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgtacgaaaggacaagagaaataaggcctacttcacaaagcgccttcccccgtaaatgatatcatctcaacttagtattatacccacacccacccaagaacagggtt

SEQ ID NO: 6 - DNA, 953 bp, Artificial
  Synthetic
aataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaa

SEQ ID NO: 7 - DNA, 21 bp, Artificial
  Synthetic
acgacggacggacggacggac

SEQ ID NO: 8 - DNA, 22 bp, Artificial
  Synthetic
taggtaaggtagagcgcggcga

SEQ ID NO: 9 - DNA, 22 bp, Artificial
  Synthetic
ttcactgtaccggccgtgcgta

SEQ ID NO: 10 - DNA, 22 bp, Artificial
  Synthetic
gcccgtcggcatgtattagctc

SEQ ID NO: 11 - DNA, 22 bp, Artificial
  Synthetic
tctagagtcaccaaagccgccg

SEQ ID NO: 12 - DNA, 21 bp, Artificial
  Synthetic
tggtcaccatggtaggcacgg

SEQ ID NO: 13 - DNA, 22 bp, Artificial
  Synthetic
ggtcgggagtgggtaatttgcg

SEQ ID NO: 14 - DNA, 22 bp, Artificial
  Synthetic
gccctccaatggatcctcgtta

SEQ ID NO: 15 - DNA, 22 bp, Artificial
  Synthetic
ccgctcccaagatccaactacg

SEQ ID NO: 16 - DNA, 22 bp, Artificial
  Synthetic
gcgggacactcagctaagagca

SEQ ID NO: 17 - DNA, 22 bp, Artificial
  Synthetic
ttcctagctgcggtatccaggc

SEQ ID NO: 18 - DNA, 22 bp, Artificial
  Synthetic
ccggtccaaggaatttcacctc

SEQ ID NO: 19 - DNA, 22 bp, Artificial
  Synthetic
tctgatcgtcttcgaacctccg

SEQ ID NO: 20 - DNA, 22 bp, Artificial
  Synthetic
gactttggtttcccggaagctg

SEQ ID NO: 21 - DNA, 22 bp, Artificial
  Synthetic
ttaagccgcaggctccactcct

SEQ ID NO: 22 - DNA, 22 bp, Artificial
  Synthetic
ctaagaacggccatgcaccacc

SEQ ID NO: 23 - DNA, 22 bp, Artificial
  Synthetic
tctaagaagttgggggacgccg

SEQ ID NO: 24 - DNA, 22 bp, Artificial
  Synthetic
taggcacacgctgagccagtca

SEQ ID NO: 25 - DNA, 22 bp, Artificial
  Synthetic
ctgggaattcctcgttcatggg

SEQ ID NO: 26 - DNA, 22 bp, Artificial
  Synthetic
ccgatccgagggcctcactaaa

SEQ ID NO: 27 - DNA, 22 bp, Artificial
  Synthetic
gctccgttaatgatccttccgc

SEQ ID NO: 28 - DNA, 21 bp, Artificial
  Synthetic
cgaacgaacgagcgagcgaac

SEQ ID NO: 29 - DNA, 20 bp, Artificial
  Synthetic
gtcgccacgtctgatctgag

SEQ ID NO: 30 - DNA, 22 bp, Artificial
  Synthetic
ctcttccctgttcactcgccgt

SEQ ID NO: 31 - DNA, 22 bp, Artificial
  Synthetic
cctcgatcagaaggacttgggc

SEQ ID NO: 32 - DNA, 21 bp, Artificial
  Synthetic
tttaccacccgctttgggctg

SEQ ID NO: 33 - DNA, 22 bp, Artificial
  Synthetic
acggtttcacgccctcttgaac

SEQ ID NO: 34 - DNA, 18 bp, Artificial
  Synthetic
cgccgggttgaatcctcc

SEQ ID NO: 35 - DNA, 21 bp, Artificial
  Synthetic
cctccggggaggaggaggagg

SEQ ID NO: 36 - DNA, 20 bp, Artificial
  Synthetic
gcaccgcctggaaatgcgcc

SEQ ID NO: 37 - DNA, 18 bp, Artificial
  Synthetic
gattcggcgagtgctgct

SEQ ID NO: 38 - DNA, 21 bp, Artificial
  Synthetic
agagcggtcgcgccgtgggag

SEQ ID NO: 39 - DNA, 20 bp, Artificial
  Synthetic
cgtggccccgagagaacctc

SEQ ID NO: 40 - DNA, 22 bp, Artificial
  Synthetic
gtccgtgtttcaagacgggtcg

SEQ ID NO: 41 - DNA, 20 bp, Artificial
  Synthetic
cgccggccttcaccttcatt

SEQ ID NO: 42 - DNA, 21 bp, Artificial
  Synthetic
tcgggtcctaacacgtgcgct

SEQ ID NO: 43 - DNA, 22 bp, Artificial
  Synthetic
tttcgcccctatacccaggtcg

SEQ ID NO: 44 - DNA, 22 bp, Artificial
  Synthetic
tcggccccaagacctctaatca

SEQ ID NO: 45 - DNA, 22 bp, Artificial
  Synthetic
actaggcactcgcattccaccc

SEQ ID NO: 46 - DNA, 21 bp, Artificial
  Synthetic
gggtctgatgagcgtcggcat

SEQ ID NO: 47 - DNA, 22 bp, Artificial
  Synthetic
gggctagttgattcggcaggtg

SEQ ID NO: 48 - DNA, 20 bp, Artificial
  Synthetic
ccctccgacgcacaccacac

SEQ ID NO: 49 - DNA, 21 bp, Artificial
  Synthetic
aggcttcaaggctcaccgcag

SEQ ID NO: 50 - DNA, 22 bp, Artificial
  Synthetic
cccttctccacttcggccttca

SEQ ID NO: 51 - DNA, 21 bp, Artificial
  Synthetic
tcgcccatctctcaggaccga

SEQ ID NO: 52 - DNA, 21 bp, Artificial
  Synthetic
ttcggggatctgaacccgact

SEQ ID NO: 53 - DNA, 22 bp, Artificial
  Synthetic
cgccctgcccttcacaaagaaa

SEQ ID NO: 54 - DNA, 21 bp, Artificial
  Synthetic
ttttcaagggccagcgagagc

SEQ ID NO: 55 - DNA, 22 bp, Artificial
  Synthetic
tgttccaacatgccagaggctg

SEQ ID NO: 56 - DNA, 21 bp, Artificial
  Synthetic
cgacccagcccttagagccaa

SEQ ID NO: 57 - DNA, 19 bp, Artificial
  Synthetic
gtggggagggagcgagcgg

SEQ ID NO: 58 - DNA, 20 bp, Artificial
  Synthetic
ctgccgccccgacccttctc

SEQ ID NO: 59 - DNA, 19 bp, Artificial
  Synthetic
cggctcgcgtccagagtcc

SEQ ID NO: 60 - DNA, 19 bp, Artificial
  Synthetic
ccgaggaggaggggggaac

SEQ ID NO: 61 - DNA, 22 bp, Artificial
  Synthetic
cgcaccagttctaagtcggctg

SEQ ID NO: 62 - DNA, 22 bp, Artificial
  Synthetic
ttgacattcagagcactgggca

SEQ ID NO: 63 - DNA, 22 bp, Artificial
  Synthetic
cgttcatccattcatgcgcgtc

SEQ ID NO: 64 - DNA, 20 bp, Artificial
  Synthetic
tgattccgccaagcccgttc

SEQ ID NO: 65 - DNA, 22 bp, Artificial
  Synthetic
cgggggcctcccacttattcta

SEQ ID NO: 66 - DNA, 21 bp, Artificial
  Synthetic
cgcctcaccgggtcagtgaaa

SEQ ID NO: 67 - DNA, 22 bp, Artificial
  Synthetic
tcaaactccccacctggcactg

SEQ ID NO: 68 - DNA, 22 bp, Artificial
  Synthetic
ccttctgctccacgggaggttt

SEQ ID NO: 69 - DNA, 21 bp, Artificial
  Synthetic
atcgtgaggccccgctttcac

SEQ ID NO: 70 - DNA, 22 bp, Artificial
  Synthetic
cgacgtcgctatgaacgcttgg

SEQ ID NO: 71 - DNA, 22 bp, Artificial
  Synthetic
gtgaacaatccaacgcttggcg

SEQ ID NO: 72 - DNA, 22 bp, Artificial
  Synthetic
tctgaacctgcggttcctctcg

SEQ ID NO: 73 - DNA, 22 bp, Artificial
  Synthetic
gcctgggcgggattctgactta

SEQ ID NO: 74 - DNA, 20 bp, Artificial
  Synthetic
gtttcccaggacgaagggca

SEQ ID NO: 75 - DNA, 22 bp, Artificial
  Synthetic
cgacccagaagcaggtcgtcta

SEQ ID NO: 76 - DNA, 22 bp, Artificial
  Synthetic
caaacccttgtgtcgagggctg

SEQ ID NO: 77 - DNA, 20 bp, Artificial
  Synthetic
tcatcgacgcacgagccgag

SEQ ID NO: 78 - DNA, 21 bp, Artificial
  Synthetic
gcaagtgcgttcgaagtgtcg

SEQ ID NO: 79 - DNA, 21 bp, Artificial
  Synthetic
agcgacgctcagacaggcgta

SEQ ID NO: 80 - DNA, 22 bp, Artificial
  Synthetic
ctcttagcggtggatcactcgg

SEQ ID NO: 81 - DNA, 24 bp, Artificial
  Synthetic
gtcgatgatcaatgtgtcctgcaa

SEQ ID NO: 82 - DNA, 22 bp, Artificial
  Synthetic
agcgacgctcagacaggcgtag

SEQ ID NO: 83 - DNA, 23 bp, Artificial
  Synthetic
actcactggaacggggatgcttg

SEQ ID NO: 84 - DNA, 22 bp, Artificial
  Synthetic
tgcgtgcttgatgcttgttcct

SEQ ID NO: 85 - DNA, 22 bp, Artificial
  Synthetic
ggcacgaaattgaccaaccctg

SEQ ID NO: 86 - DNA, 22 bp, Artificial
  Synthetic
acgccggcttctattgacttgg

SEQ ID NO: 87 - DNA, 23 bp, Artificial
  Synthetic
ggtcttagctattgtgtgttcag

SEQ ID NO: 88 - DNA, 22 bp, Artificial
  Synthetic
tgtggctcgtagtgttctggcg

SEQ ID NO: 89 - DNA, 22 bp, Artificial
  Synthetic
agagggatatgaagcaccgcca

SEQ ID NO: 90 - DNA, 22 bp, Artificial
  Synthetic
agaggtggtgaggttgatcggg

SEQ ID NO: 91 - DNA, 22 bp, Artificial
  Synthetic
tcagggtttgctgaagatggcg

SEQ ID NO: 92 - DNA, 22 bp, Artificial
  Synthetic
ttgccacctcatgggctacacc

SEQ ID NO: 93 - DNA, 22 bp, Artificial
  Synthetic
ggcggtgtgtacgcgcttcagg

SEQ ID NO: 94 - DNA, 23 bp, Artificial
  Synthetic
ccatgttacgacttgtctcctct

SEQ ID NO: 95 - DNA, 22 bp, Artificial
  Synthetic
gtctggtagtaaggtggagtgg

SEQ ID NO: 96 - DNA, 22 bp, Artificial
  Synthetic
ttcatctttcccttgcggtact

SEQ ID NO: 97 - DNA, 23 bp, Artificial
  Synthetic
ctgttcttaggtagctcgtctgg

SEQ ID NO: 98 - DNA, 22 bp, Artificial
  Synthetic
aaccagctatcaccaggctcgg

SEQ ID NO: 99 - DNA, 23 bp, Artificial
  Synthetic
cctagtgtccaaagagctgttcc

SEQ ID NO: 100 - DNA, 22 bp, Artificial
  Synthetic
tgggtgttgagcttgaacgctt

SEQ ID NO: 101 - DNA, 22 bp, Artificial
  Synthetic
tctgacgcaggcttatgcggag

SEQ ID NO: 102 - DNA, 22 bp, Artificial
  Synthetic
tgagcatgcctgtgttgggttg

SEQ ID NO: 103 - DNA, 22 bp, Artificial
  Synthetic
ccctcgtggagccattcataca

SEQ ID NO: 104 - DNA, 22 bp, Artificial
  Synthetic
tcgtcttgctgtgttatgcccg

SEQ ID NO: 105 - DNA, 22 bp, Artificial
  Synthetic
actgctcggaggttgggttctg

SEQ ID NO: 106 - DNA, 22 bp, Artificial
  Synthetic
agggtaacttgttccgttggtc

SEQ ID NO: 107 - DNA, 22 bp, Artificial
  Synthetic
ttaatagcggctgcaccatcgg

SEQ ID NO: 108 - DNA, 22 bp, Artificial
  Synthetic
ctccggtctgaactcagatcac

SEQ ID NO: 109 - DNA, 22 bp, Artificial
  Synthetic
ggggaaggcgctttgtgaagta

SEQ ID NO: 110 - DNA, 22 bp, Artificial
  Synthetic
accctgttcttgggtgggtgtg

SEQ ID NO: 111 - DNA, 22 bp, Artificial
  Synthetic
gacctcagatcagacgtggcga

SEQ ID NO: 112 - DNA, 20 bp, Artificial
  Synthetic
gtcgggttgcttgggaatgc

SEQ ID NO: 113 - DNA, 18 bp, Artificial
  Synthetic
tccgcccggaggattcaa

SEQ ID NO: 114 - DNA, 18 bp, Artificial
  Synthetic
gcatttccaggcggtgcg

SEQ ID NO: 115 - DNA, 18 bp, Artificial
  Synthetic
agcagcactcgccgaatc

SEQ ID NO: 116 - DNA, 18 bp, Artificial
  Synthetic
acccctcccacggcgcga

SEQ ID NO: 117 - DNA, 21 bp, Artificial
  Synthetic
cacggaccaaggagtctaaca

SEQ ID NO: 118 - DNA, 22 bp, Artificial
  Synthetic
cgaaagatggtgaactatgcct

SEQ ID NO: 119 - DNA, 22 bp, Artificial
  Synthetic
ccgaaacgatctcaacctattc

SEQ ID NO: 120 - DNA, 22 bp, Artificial
  Synthetic
cacctgccgaatcaactagccc

SEQ ID NO: 121 - DNA, 19 bp, Artificial
  Synthetic
ccacgcctcctcccctcct

SEQ ID NO: 122 - DNA, 21 bp, Artificial
  Synthetic
gaacagcagttgaacatgggt

SEQ ID NO: 123 - DNA, 22 bp, Artificial
  Synthetic
cggggagagttctcttttcttt

SEQ ID NO: 124 - DNA, 22 bp, Artificial
  Synthetic
caatgtaggtaagggaagtcgg

SEQ ID NO: 125 - DNA, 20 bp, Artificial
  Synthetic
ctctctctctcccccgctcc

SEQ ID NO: 126 - DNA, 20 bp, Artificial
  Synthetic
tcccgtggatcgccccagct

SEQ ID NO: 127 - DNA, 20 bp, Artificial
  Synthetic
ggcggggcggggcggttcgt

SEQ ID NO: 128 - DNA, 22 bp, Artificial
  Synthetic
cctagcagccgacttagaactg

SEQ ID NO: 129 - DNA, 22 bp, Artificial
  Synthetic
tctcttaaggtagccaaatgcc

SEQ ID NO: 130 - DNA, 22 bp, Artificial
  Synthetic
ctactatccagcgaaaccacag

SEQ ID NO: 131 - DNA, 22 bp, Artificial
  Synthetic
cggggaaagaagaccctgttga

SEQ ID NO: 132 - DNA, 21 bp, Artificial
  Synthetic
ctcagggaggacagaaacctc

SEQ ID NO: 133 - DNA, 22 bp, Artificial
  Synthetic
gatgtcggctcttcctatcatt

SEQ ID NO: 134 - DNA, 22 bp, Artificial
  Synthetic
actaatagggaacgtgagctgg

SEQ ID NO: 135 - DNA, 22 bp, Artificial
  Synthetic
ctaagtcagaatcccgcccagg

SEQ ID NO: 136 - DNA, 22 bp, Artificial
  Synthetic
tcgccgcgctctaccttaccta

SEQ ID NO: 137 - DNA, 22 bp, Artificial
  Synthetic
gagctaatacatgccgacgggc

SEQ ID NO: 138 - DNA, 22 bp, Artificial
  Synthetic
aacgtctgccctatcaactttc

SEQ ID NO: 139 - DNA, 21 bp, Artificial
  Synthetic
ggccctgtaattggaatgagt

SEQ ID NO: 140 - DNA, 22 bp, Artificial
  Synthetic
tgctcttagctgagtgtcccgc

SEQ ID NO: 141 - DNA, 22 bp, Artificial
  Synthetic
tagaggtgaaattccttggacc

SEQ ID NO: 142 - DNA, 23 bp, Artificial
  Synthetic
tgaaacttaaaggaattgacgga

SEQ ID NO: 143 - DNA, 21 bp, Artificial
  Synthetic
cacggacaggattgacagatt

SEQ ID NO: 144 - DNA, 23 bp, Artificial
  Synthetic
cccaacttcttagagggacaagt

SEQ ID NO: 145 - DNA, 21 bp, Artificial
  Synthetic
ctgtgatgcccttagatgtcc

SEQ ID NO: 146 - DNA, 23 bp, Artificial
  Synthetic
atgaacgaggaattcccagtaag

SEQ ID NO: 147 - DNA, 21 bp, Artificial
  Synthetic
acctgcggaaggatcattaac

SEQ ID NO: 148 - DNA, 22 bp, Artificial
  Synthetic
cccactccaccttactaccaga

SEQ ID NO: 149 - DNA, 22 bp, Artificial
  Synthetic
caaggagagccaaagctaagac

SEQ ID NO: 150 - DNA, 22 bp, Artificial
  Synthetic
agaaccctctaaatccccttgt

SEQ ID NO: 151 - DNA, 22 bp, Artificial
  Synthetic
caaagaggaacagctctttgga

SEQ ID NO: 152 - DNA, 22 bp, Artificial
  Synthetic
taactgaactcctcacacccaa

SEQ ID NO: 153 - DNA, 22 bp, Artificial
  Synthetic
acaggcatgctcataaggaaag

SEQ ID NO: 154 - DNA, 22 bp, Artificial
  Synthetic
accgtgcaaaggtagcataatc

SEQ ID NO: 155 - DNA, 22 bp, Artificial
  Synthetic
agcaagacgagaagaccctatg

SEQ ID NO: 156 - DNA, 22 bp, Artificial
  Synthetic
ttcaccagtcaaagcgaactac

SEQ ID NO: 157 - DNA, 22 bp, Artificial
  Synthetic
gatccaataacttgaccaacgg

SEQ ID NO: 158 - DNA, 22 bp, Artificial
  Synthetic
aagattacacatgcaagcatcc

SEQ ID NO: 159 - DNA, 22 bp, Artificial
  Synthetic
gtcacacgattaacccaagtca

SEQ ID NO: 160 - DNA, 22 bp, Artificial
  Synthetic
gataccccactatgcttagccc

SEQ ID NO: 161 - DNA, 22 bp, Artificial
  Synthetic
ttctgtaatcgataaaccccga

SEQ ID NO: 162 - DNA, 21 bp, Artificial
  Synthetic
gatcaacctcaccacctcttg

SEQ ID NO: 163 - DNA, 22 bp, Artificial
  Synthetic
ggctacattttctaccccagaa

SEQ ID NO: 164 - DNA, 22 bp, Artificial
  Synthetic
gactcttagcggtggatcactc

SEQ ID NO: 165 - DNA, 20 bp, Artificial
  Synthetic
gtctacggccataccaccct

SEQ ID NO: 166 - DNA, 759 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 167 - DNA, 775 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 168 - DNA, 773 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 169 - DNA, 760 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccgccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 170 - DNA, 767 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 171 - DNA, 775 bp, Artificial; identical to SEQ ID NO: 167
  Synthetic

SEQ ID NO: 172 - DNA, 769 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 173 - DNA, 764 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccgccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 174 - DNA, 773 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccgccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 175 - DNA, 770 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 176 - DNA, 774 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccgccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgcccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 177 - DNA, 770 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccgccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 178 - DNA, 769 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgcccccccccacccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcgggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 179 - DNA, 761 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgccccccccccaccccacgtctcgtcgcgcgcgcgtccgctgggggcggggagcggtcgggcggcggcggtcggcgggcggcggggcggggcggttcgtccccccgccctacccccccggccccgtccgccccccccttcccccctcctcctcggcgcgcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 180 - DNA, 759 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgcccccccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcgggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 181 - DNA, 759 bp, Artificial; identical to SEQ ID NO: 180
  Synthetic

SEQ ID NO: 182 - DNA, 761 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgcccccccccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 183 - DNA, 827 bp, Artificial
  Synthetic
tctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgcgcgccccccccacgcccggggcacccccctcgcggccctcccccgccccacccgcgcgcgccgctcgctccctccccaccccgcgccctctctctctctctctcccccgctccccgtcctcccccctccccgggggagcgccgcgtgggggcgcggcggggggagaagggtcggggcgccaggggccgcgcggcggccgccggggcggccggcgggggcaggtccccgcgaggggggccccgggcacccggggggccggcggcggcgcggactctggacgcgagccgggcccttcccgtggatcgccccagctgcggcgggcgtcgcggccgcccccggggagcccggcggcggcgcggcgcgccccccacccccaccccacgtctcggtcgcgcgcgcgtccgctgggggcgggagcggtcgggcggcggcggtcggcgggcggcggggcggggcggttcgtccccccgccctacccccccggccccgtccgcccccccttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccgactgtttaattaaaacaaa

SEQ ID NO: 184 - DNA, 771 bp, Artificial
  Synthetic
ggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccacgcgcgccgctcgctccctccccaccccgcgccctctctctctctctctcccccgctccccgtcctcccccctccccgggggagcgccgcgtgggggcggcggcggggggagaagggtcggggcgccaggggccggcggcggcccgccgggggccccggcggcggggcacggtcccccggaggggcccgggcacccggggggccggcggcggcggcgactctggacgcgagccgggcccttcccgtggatcgccccagctgcggcggcgtcgcggccgcccccggggagcccggcggccggcgcgcccccccccaccccccaccccacgtctcgtcgcgcgcgcgtccgctgggggcggggagcggtcgggcggcggcggtcggcgggcggcggggcggggcggttcgtccccccgccctacccccccggccccgtccgcccccccttcccccctcctcctcggcgcgcggcggcggcggcgggcgaggcggcggaggggccgcgggccggtcccccccgccgggtccgcccccggggccgcggttccgcgcgcgcctcgcctcggccggcgcctagcagccgacttagaactggtgcggaccaggggaatccga

SEQ ID NO: 185 - DNA, 23 bp, Artificial
  Synthetic
cttgccgtcccaagcaatggatg

SEQ ID NO: 186 - DNA, 24 bp, Artificial
  Synthetic
caataggtgtgcgtcagaagcacc

SEQ ID NO: 187 - DNA, 24 bp, Artificial
  Synthetic
ggtctcacagtgttgcccaggctg

SEQ ID NO: 188 - DNA, 20 bp, Artificial
  Synthetic
ggaugaacguaggaagcuug

SEQ ID NO: 189 - DNA, 26 bp, Artificial
  Synthetic
ggaattaatacgactcactatagggn

SEQ ID NO: 190 - DNA, 44 bp, Artificial
  Synthetic
gcgcgaaattaaccctcactaaagcctacttgcatccttcgaac

SEQ ID NO: 191 - DNA, 21 bp, Artificial
  Synthetic
agcaacccgactccgggaaga

SEQ ID NO: 192 - DNA, 20 bp, Artificial
  Synthetic
gacccgccgccgggttgaat

SEQ ID NO: 193 - DNA, 18 bp, Artificial
  Synthetic
cgcaccgcctggaaatgc

SEQ ID NO: 194 - DNA, 18 bp, Artificial
  Synthetic
gattcggcgagtgctgct

SEQ ID NO: 195 - DNA, 18 bp, Artificial
  Synthetic
tggccccgagagaacctc

SEQ ID NO: 196 - DNA, 20 bp, Artificial
  Synthetic
cgtgtttcaagacgggtcgg

SEQ ID NO: 197 - DNA, 22 bp, Artificial
  Synthetic
ccaggcatagttcaccatcttt

SEQ ID NO: 198 - DNA, 22 bp, Artificial
  Synthetic
gccccaagacctctaatcattc

SEQ ID NO: 199 - DNA, 23 bp, Artificial
  Synthetic
tgttacacactccttagcggatt

SEQ ID NO: 200 - DNA, 20 bp, Artificial
  Synthetic
aggcttcaaggctcaccgca

SEQ ID NO: 201 - DNA, 22 bp, Artificial
  Synthetic
caggaccgactgacccatgttc

SEQ ID NO: 202 - DNA, 22 bp, Artificial
  Synthetic
ctgcccttcacaaagaaaagag

SEQ ID NO: 203 - DNA, 22 bp, Artificial
  Synthetic
gccgacttcccttacctacatt

SEQ ID NO: 204 - DNA, 21 bp, Artificial
  Synthetic
gagagagagagagagagggcg

SEQ ID NO: 205 - DNA, 20 bp, Artificial
  Synthetic
agctggggcgatccacggga

SEQ ID NO: 206 - DNA, 20 bp, Artificial
  Synthetic
ccgaggaggaggggggaacg

SEQ ID NO: 207 - DNA, 22 bp, Artificial
  Synthetic
cgcaccagttctaagtcggctg

SEQ ID NO: 208 - DNA, 22 bp, Artificial
  Synthetic
gacagtgggaatctcgttcatc

SEQ ID NO: 209 - DNA, 21 bp, Artificial
  Synthetic
gggcctcccacttattctaca

SEQ ID NO: 210 - DNA, 22 bp, Artificial
  Synthetic
agagtagtggtatttcaccggc

SEQ ID NO: 211 - DNA, 21 bp, Artificial
  Synthetic
ctcgccttaggacacctgcgt

SEQ ID NO: 212 - DNA, 22 bp, Artificial
  Synthetic
aatgataggaagagccgacatc

SEQ ID NO: 213 - DNA, 22 bp, Artificial
  Synthetic
cgttcagtcataatcccacaga

SEQ ID NO: 214 - DNA, 22 bp, Artificial
  Synthetic
ggattctgacttagaggcgttc

SEQ ID NO: 215 - DNA, 21 bp, Artificial
  Synthetic
gtttagcgccaggttccccac

SEQ ID NO: 216 - DNA, 22 bp, Artificial
  Synthetic
gcccgtcggcatgtattagctc

SEQ ID NO: 217 - DNA, 22 bp, Artificial
  Synthetic
gaatcgaaccctgattccccgt

SEQ ID NO: 218 - DNA, 22 bp, Artificial
  Synthetic
tggactcattccaattacaggg

SEQ ID NO: 219 - DNA, 22 bp, Artificial
  Synthetic
gggacactcagctaagagcatc

SEQ ID NO: 220 - DNA, 21 bp, Artificial
  Synthetic
gccggtccaaggaatttcacc

SEQ ID NO: 221 - DNA, 23 bp, Artificial
  Synthetic
gtttcagctttgcaaccatactc

SEQ ID NO: 222 - DNA, 22 bp, Artificial
  Synthetic
agttagcatgccagagtctcgt

SEQ ID NO: 223 - DNA, 22 bp, Artificial
  Synthetic
catctaagggcatcacagacct

SEQ ID NO: 224 - DNA, 22 bp, Artificial
  Synthetic
agcttatgacccgcacttactg

SEQ ID NO: 225 - DNA, 22 bp, Artificial
  Synthetic
ctcactaaaccatccaatcggt

SEQ ID NO: 226 - DNA, 22 bp, Artificial
  Synthetic
cctacggaaaccttgttacgac

SEQ ID NO: 227 - DNA, 22 bp, Artificial
  Synthetic
acgcccttctttctctctctct

SEQ ID NO: 228 - DNA, 22 bp, Artificial
  Synthetic
ggtcttagctttggctctcctt

SEQ ID NO: 229 - DNA, 22 bp, Artificial
  Synthetic
agtgtccaaagagctgttcctc

SEQ ID NO: 230 - DNA, 22 bp, Artificial
  Synthetic
attgggtgtgaggagttcagtt

SEQ ID NO: 231 - DNA, 22 bp, Artificial
  Synthetic
cggaggagaatgttttcatgtt

SEQ ID NO: 232 - DNA, 22 bp, Artificial
  Synthetic
ctttccttatgagcatgcctgt

SEQ ID NO: 233 - DNA, 22 bp, Artificial
  Synthetic
taagagacagctgaaccctcgt

SEQ ID NO: 234 - DNA, 22 bp, Artificial
  Synthetic
gcaggtttggtagtttaggacc

SEQ ID NO: 235 - DNA, 22 bp, Artificial
  Synthetic
ctagggtaacttgttccgttgg

SEQ ID NO: 236 - DNA, 22 bp, Artificial
  Synthetic
ggtagatagaaaccgacctgga

SEQ ID NO: 237 - DNA, 22 bp, Artificial
  Synthetic
ctcttgtcctttcgtacaggga

SEQ ID NO: 238 - DNA, 22 bp, Artificial
  Synthetic
tgacttgggttaatcgtgtgac

SEQ ID NO: 239 - DNA, 21 bp, Artificial
  Synthetic
gtagtgttctggcgagcagtt

SEQ ID NO: 240 - DNA, 22 bp, Artificial
  Synthetic
cttgcgcttactttgtagcctt

SEQ ID NO: 241 - DNA, 22 bp, Artificial
  Synthetic
atccaccttcgacccttaagtt

SEQ ID NO: 242 - DNA, 22 bp, Artificial
  Synthetic
gccctgttcaactaagcactct

SEQ ID NO: 243 - DNA, 23 bp, Artificial
  Synthetic
tgcactttccagtacacttacca

SEQ ID NO: 244 - DNA, 23 bp, Artificial
  Synthetic
ttcgaagtgtcgatgatcaatgt

SEQ ID NO: 245 - DNA, 22 bp, Artificial
  Synthetic
cggtctcccatccaagtactaa

SEQ ID NO: 246 - DNA, 23 bp, Artificial
  Synthetic
gacccaggtccagatgaagctcc

SEQ ID NO: 247 - DNA, 25 bp, Artificial
  Synthetic
caaaacaccagtgcaggccaacttg

SEQ ID NO: 248 - DNA, 23 bp, Artificial
  Synthetic
ttgtaatcccagcactctgggag

(19) United States
(12) Patent Application Publication
(10) Pub. No.: US 2009/0137415 A1
(43) Pub. Date: May 28, 2009

(54) SUBTRACTIVE SEPARATION AND AMPLIFICATION OF NON-RIBOSOMAL TRANSCRIBED RNA (nrRNA)

(75) Inventors: Wadiha Freije, Forest Park, IL (US); Igor Brikun, Forest Park, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: EUCLID DIAGNOSTICS LLC, Chicago, IL (US)

(21) Appl. No.: 11/997,993

(22) PCT Filed: Aug. 4, 2006
(86) PCT No.: PCT/US06/30721

§ 371 Date: Feb. 5, 2008

Related U.S. Application Data
(60) Provisional application No. 60/705,964, filed on Aug. 5, 2005.

(51) Int. Cl. C40B 30/04; C07H 1/00; C12P 19/34; C12M 1/00; C12Q 1/68

(52) U.S. Cl. 506/9; 536/25.4; 435/91.1; 435/6; 435/287.2

(57) ABSTRACT

The invention provides a method of separating non-ribosomal transcribed RNA (nrRNA) fragments from ribosomal RNA (rRNA) and rRNA fragments. The method comprises (i) providing a sample comprising rRNA, rRNA fragments, and nrRNA fragments, and (ii) providing a plurality of probes. The probes hybridize to RNA targeting sequences of at least 50% of the contiguous regions of the rRNA and to rRNA fragments comprising the rRNA targeting sequences. The method further comprises (iii) adding the plurality of probes to the sample, (iv) hybridizing the probes to the rRNA and rRNA fragments to form rRNA-probe complexes and rRNA fragment-probe complexes, and (v) separating the rRNA-probe complexes and rRNA fragment-probe complexes. The invention also provides a method of amplifying an nrRNA fragment, a method of analyzing nrRNA expression, a method of determining the level of nrRNA in a sample, and a kit and system useful in any of the foregoing methods.

### CROSS-REFERENCE TO RELATED APPLICATIONS

This patent application claims the benefit of U.S. Provisional Patent Application No. 60/705,964, filed Aug. 5, 2005.

### TECHNICAL FIELD OF THE INVENTION

The invention relates to methods comprising separating messenger RNA (mRNA) and/or non-coding RNA (ncRNA), referred to collectively herein as non-ribosomal transcribed RNA (nrRNA), from a sample, such as a sample of total RNA. The invention also relates to kits and systems for use in such methods.

### BACKGROUND OF THE INVENTION

Most mRNA isolation kits rely on the oligo dT-mediated purification of mRNA. This method is disadvantageous when mRNA is degraded and, hence, fragmented. The oligo dT only binds to the polyA tail of mRNA and, therefore, only purifies intact mRNA and molecules of mRNA that contain polyA tails. Molecules of mRNA that do not contain polyA tails are not purified. For this reason, when performing expression analysis using RNA isolated from fixed tissues, scientists have relied on the reverse transcription of total RNA using random hexamers even though over 95% of the RNA present in the sample is ribosomal (Godfrey et al., J. Molec. Diagnostics 2(2): 84-91 (2000)). This approach is disadvantageous because it is not sensitive and does not enable optimal detection of mRNA present in low copy numbers.

To overcome this limitation, scientists have used oligonucleotide primers designed to anneal to a plurality of genes for the reverse transcription reaction (see, for example, U.S. Patent Application Publication Nos. 2004/0259105 and 2005/0095634). Using this approach, only the expression level of a limited number of genes can be determined. Furthermore, only small portions of those genes, which are upstream of the primer site, can be analyzed, since the majority of the RNA fragments are less than 500 base pairs in length. In addition, if the fragments are very short, such as less than 50 base pairs, it may be extremely difficult to design an assay to determine expression levels, whether by polymerase chain reaction, ligation-mediated amplification, or microarray analysis, for example.

Murphy and Whitley (U.S. Patent Application Publication No. 2003/0175709 and International Patent Application Publication No. WO 03/054162) have proposed the use of a bridging nucleic acid, which includes at least one targeting region and at least one bridging region, and a capture nucleic acid, which includes a capture region and a non-reacting structure. The targeting region is complementary to a targeted region of a targeted nucleic acid to be removed. The bridging region is complementary to the capture region. The nonreacting structure is a compound that does not react with a nucleic acid. The bridging nucleic acid can include up to 10 or more targeting regions that are complementary to different, non-overlapping targeted regions from the same or different targeted nucleic acids. This method is disadvantageous in that the use of multiple targeting regions on a single bridging nucleic acid can decrease the efficiency of hybridization between the targeting regions and the complementary targeted regions. In addition, the use of multiple targeting regions on a single bridging nucleic acid can interfere with hybridization between the bridging region of the bridging nucleic acid and the capture region of the capture nucleic acid. These effects also can decrease efficiency of recovery of non-ribosomal RNA. The methods disclosed by Murphy and Whitley also result in increased background hybridization due to the length of the oligonucleotides used.

The invention seeks to overcome the disadvantages of the currently available isolation kits by providing a method that enables the purification of nrRNA fragments that do not contain polyA tails as well as intact mRNA and/or ncRNA. This and other objects and advantages of the invention, as well as additional inventive features, will become apparent from the detailed description provided herein.

### BRIEF SUMMARY OF THE INVENTION

The invention provides a method of separating non-ribosomal transcribed RNA (nrRNA) fragments from ribosomal RNA (rRNA) and rRNA fragments. The method comprises (i) providing a sample comprising rRNA, rRNA fragments, and nrRNA fragments, wherein the rRNA comprises multiple contiguous regions of about 100 base pairs. Each contiguous region comprises an rRNA targeting sequence, and the rRNA fragments comprise one or more of the rRNA targeting sequences. The method further comprises (ii) providing a plurality of probes. The probes hybridize to (a) different RNA targeting sequences of at least 50% of the contiguous regions of the rRNA and (b) rRNA fragments comprising the rRNA targeting sequences hybridized to by the probes in the contiguous regions of the rRNA. The method further comprises (iii) adding the plurality of probes to the sample, (iv) hybridizing the probes to the rRNA and rRNA fragments to form rRNA-probe complexes and rRNA fragment-probe complexes, and (v) separating the rRNA-probe complexes and rRNA fragment-probe complexes from the sample, thereby separating nrRNA fragments from rRNA and rRNA fragments.

The invention further provides a method of amplifying a cDNA complementary to an nrRNA fragment. The method comprises (i) separating an nrRNA fragment from rRNA and rRNA fragments according to the inventive method, wherein the nrRNA fragment comprises a 5′ end. The method also comprises (ii) ligating a first oligonucleotide to the 5′ end of the nrRNA fragment, (iii) hybridizing to the nrRNA fragment a second oligonucleotide, (iv) extending a DNA strand from the second oligonucleotide to generate a first cDNA, and (v) amplifying the first cDNA, thereby amplifying a cDNA complementary to an nrRNA fragment.

In addition, the invention provides a method of amplifying nrRNA fragments. The method comprises (i) separating nrRNA fragments from rRNA and rRNA fragments according to the inventive method. The method further comprises (ii) amplifying the nrRNA fragments. Alternatively, the method further comprises (ii) ligating a first oligonucleotide to the 5′ end of the nrRNA fragment; (iii) hybridizing to the nrRNA fragment a second oligonucleotide; and (iv) extending a DNA strand from the second oligonucleotide to generate a first cDNA having a 5′ end and a 3′ end. The method then comprises (v) degrading the nrRNA fragment and, optionally, removing any free oligonucleotides and/or free RNA fragments; (vi) synthesizing a complementary strand to the first cDNA; (vii) optionally purifying the double-stranded DNA copy; and (viii) transcribing RNA from the double-stranded DNA copy. Synthesizing a complementary strand to the first cDNA in step (vi) comprises (a) contacting the first cDNA with a third oligonucleotide such that the third oligonucleotide hybridizes to the 3′ end of the first cDNA and (b) extending the complementary strand from the third oligonucleotide, whereupon a double-stranded DNA copy of the nrRNA fragment is generated. The third oligonucleotide hybridizes to at least a portion of the first oligonucleotide and optionally contains an RNA polymerase promoter sequence.

Still further provided by the invention is a method of analyzing nrRNA expression. The method comprises (i) separating nrRNA fragments from rRNA and rRNA fragments according to the inventive method and (ii) analyzing nrRNA expression. Additionally, the invention provides a method of determining the level of nrRNA in a sample. The method comprises (i) separating nrRNA fragments from rRNA and rRNA fragments according to the inventive method and (ii) labeling the separated nrRNA fragments with a detectable label. The method of determining the level of nrRNA in a sample further comprises (iii) providing an array comprising a collection of fixed DNAs that hybridize to a plurality of target sequences located within an expression sequence of a gene of interest. The fixed DNAs comprise from about 15 nucleotides to about 750 nucleotides, and at least one target sequence is interspersed approximately every 500 base pairs of the expression sequence. The method additionally comprises (iv) applying the labeled nrRNA fragments to the array under hybridization conditions; (v) optionally removing unbound and nonspecifically bound nrRNA fragments from the array; (vi) detecting the labeled nrRNA fragments that have specifically annealed to the fixed DNAs in the array; and (vii) quantitating the level of labeled nrRNA fragments detected in step (vi) to determine the level of nrRNA in the sample.

The invention also provides a kit for separating nrRNA fragments from a sample comprising rRNA and rRNA fragments. The kit comprises a plurality of probes that hybridize to (a) different rRNA target sequences of at least 50% of contiguous regions of about 100 base pairs of the rRNA, which contiguous regions comprise the rRNA targeting sequences and (b) rRNA fragments comprising the rRNA targeting sequences hybridized to by the probes in the contiguous regions of the rRNA. Preferably, the kit also comprises instructions.

Likewise, the invention provides a system for separating nrRNA fragments from a sample comprising rRNA and rRNA fragments. The system comprises a device adapted for the separation of nucleic acids by hybridization. The system also comprises a plurality of probes that hybridize to (a) different rRNA target sequences of at least 50% of contiguous regions of about 100 base pairs of the rRNA, which contiguous regions comprise the rRNA targeting sequences, and (b) rRNA fragments comprising the rRNA targeting sequences hybridized to by the probes in the contiguous regions of the rRNA. The plurality of probes is positioned within the device to separate the rRNA and rRNA fragments from nrRNA.

### BRIEF DESCRIPTION OF THE FIGURES

FIGS. 1A-1D show the nucleotide sequences of the 28S (SEQ ID NO: 1), 18S (SEQ ID NO: 2), 5.8S (SEQ ID NO: 3), 5S (SEQ ID NO: 4), 16S (SEQ ID NO: 5), and 12S (SEQ ID NO: 6) rRNAs. Sequences are presented from left to right, top to bottom, in accordance with convention.

FIGS. 2A-2D show a set of oligonucleotides (SEQ ID NOs: 7-110) that can hybridize to rRNA and are suitable for use in the method. The “Start” positions are given relative to the sequences of FIGS. 1A-1D.

FIGS. 3A-3C show a set of forward and reverse primers (SEQ ID NOs: 111-165 and 191-245) that can amplify fragments corresponding to six rRNA species. The “Left” and “Right” designations indicate forward and reverse primers, respectively.

FIGS. 4A-4M compare 28S rRNA sequences and provide the consensus sequence (SEQ ID NOs: 166-184).

FIG. 5 is a diagram illustrating the preparation of double-stranded cDNA for amplification.

### DETAILED DESCRIPTION OF THE INVENTION

The invention is predicated on the discovery that the use of an exhaustive set of probes enables the efficient removal of ribosomal RNA (rRNA) (i.e., intact rRNA and/or rRNA fragments) from a sample of total RNA. In doing so, the invention enables the separation and/or purification and/or enrichment of non-ribosomal transcribed RNA (nrRNA) fragments that do not contain polyA tails, such as are commonly found in degraded RNA. The invention does not suffer from the disadvantages attendant the use of currently available methods.

The invention provides a method of separating nrRNA, such as messenger RNA (mRNA) and non-coding RNA (ncRNA), from a sample, such as a sample comprising total RNA. The inventive method can be performed on any sample suitable for separation of RNA species. Indeed, any suitable sample of, for example, total RNA can be used. Preferably, degradation of RNA in the sample has been minimized. However, the invention is advantageous in that it can be used on degraded RNA, such as RNA recovered from preserved tissue samples, e.g., paraffin-embedded tissue samples, biopsies, surgical specimens, and any other source containing degraded RNA. Such RNA is usually less than 500 base pairs in size and may not represent the entire population of mRNA species present in original tissue samples. The invention also permits the recovery of ncRNA species that are present in a total RNA sample, such as small interfering RNA (siRNA) and small nuclear RNA (snRNA). Researchers have great difficulty in efficiently isolating such small RNA species using currently known methods.

The method comprises (i) removing rRNA from the sample of total RNA by hybridizing the rRNA with a plurality of probes comprising at least one probe that can hybridize to a complementary sequence within about 100 contiguous base pairs of the rRNA to be removed such that at least one probe hybridizes to a complementary sequence within at least about every 100 contiguous base pairs of the rRNA to be removed, and (ii) purifying the nrRNA.

Alternatively, the invention provides a method of separating nrRNA fragments from rRNA and rRNA fragments. The method comprises: (i) providing a sample comprising rRNA, rRNA fragments, and nrRNA fragments; (ii) providing a plurality of probes; (iii) adding the plurality of probes to the sample; (iv) hybridizing the probes to the rRNA and rRNA fragments to form rRNA-probe complexes and rRNA fragment-probe complexes, and (v) separating the rRNA-probe complexes and rRNA fragment-probe complexes from the sample (i.e., separating the hybridized RNA from the sample of RNA). Optionally, simultaneously with or subsequently to removing rRNA from the sample (e.g., the sample of total RNA), the method can further comprise removing any other RNA that is not of interest, such as unwanted mRNA species that are abundantly expressed or tRNA(s). It will be understood that “separating” the rRNA-probe complexes and/or rRNA fragment-probe complexes (or any other RNA) from the sample does not require complete elimination of rRNA or rRNA fragments from the sample. If desired, additional rounds of hybridization in accordance with the inventive method can be performed to further remove unwanted RNA. For example, the method can be performed on a sample of total RNA to achieve a sample comprising no more than 20% rRNA. Preferably, the method yields a sample or composition comprising no more than 10% rRNA, and more preferably the resulting composition comprises no more than 5% rRNA. Most preferably, the composition resulting from the inventive method comprises no more than 1% rRNA. The inventive method preferably further comprises (vi) recovering and purifying the nrRNA fragments. In view of the above, the method is particularly suited for enriching a composition for nrRNA and fragments thereof by removing unwanted RNAs, such as rRNA and rRNA fragments.

The invention comprises employing a plurality of probes to saturate unwanted rRNA in a sample, thereby providing multiple “handles” for removing the unwanted material using any suitable separation technique. In this regard, rRNA and rRNA fragments can be conceptually divided into multiple contiguous regions of about 100 base pairs. For example, an rRNA or rRNA fragment that comprises 1000 base pairs comprises ten contiguous regions of about 100 base pairs. Each contiguous region comprises one or more RNA targeting sequences. Preferably, two or more of the multiple contiguous regions comprise different rRNA targeting sequences. An “rRNA targeting sequence” refers to a nucleotide sequence within the rRNA or rRNA fragment that is complementary to, and thereby hybridizes with, one or more probes of the plurality. The rRNA fragments of the sample desirably comprise one or more of the rRNA targeting sequences.

A “plurality of probes” is a collection of molecules, such as oligonucleotides, that bind rRNA target sequences within the rRNA or rRNA fragments of the sample. Preferably, the probes hybridize to (a) different RNA targeting sequences of at least 50% of the contiguous regions of the rRNA and (b) rRNA fragments comprising the rRNA targeting sequences hybridized to by the probes in the contiguous regions of the rRNA. In other words, if an rRNA comprises ten contiguous regions of about 100 base pairs, the probes hybridize to RNA targeting sequences in at least five of the ten contiguous regions. By “different” RNA targeting sequences is meant separate, individual RNA targeting sequences, which may or may not be unique. Desirably, multiple probes hybridize over the length of the unwanted RNA to ensure efficient removal. For example, the probes of the inventive method can hybridize to rRNA targeting sequences of at least 60% of the contiguous regions (e.g., the probes hybridize to rRNA targeting sequences of at least 70% of the contiguous regions). Even more preferably, the probes hybridize to rRNA targeting sequences of at least 80% of the contiguous regions (e.g., the probes hybridize to rRNA targeting sequences of at least 90% of the contiguous regions). Still more preferably, the probes hybridize to rRNA targeting sequences of at least 95% of the contiguous regions. Even more preferably, the probes hybridize to rRNA targeting sequences of all of the contiguous regions (i.e., rRNA targeting sequences located in each and every contiguous region).

In this regard, rRNA and fragments thereof and, optionally, any other RNA that is not of interest, can be removed from a sample, e.g., a sample of total RNA, by hybridizing the RNA to a plurality of probes comprising, for example, at least one tagged oligonucleotide (i.e., DNA or RNA, including modified base analogs), tagged single-stranded nucleic acid molecule (i.e., DNA or RNA), or tagged DNA mimic, such as a locked nucleic acid (LNA; Koshkin et al., Tetrahedron 54: 3607-3630 (1998); Koshkin et al., JACS 120: 13252-13253 (1998); and Wahlestedt et al., PNAS 97: 5633-5638 (2000)) and/or a DNA analog with a non-phosphodiester backbone (such as a peptide nucleic acid (PNA; Nielson, Molec. Biotech. 26: 233-248 (2004); and Egholm et al., Nature 365(6446): 566-568 (1993)) or morpholino derivatives (Summerton, BioChim. Biophys. Acta 1489:141-158 (1999)), which is complementary to the RNA that is to be removed.

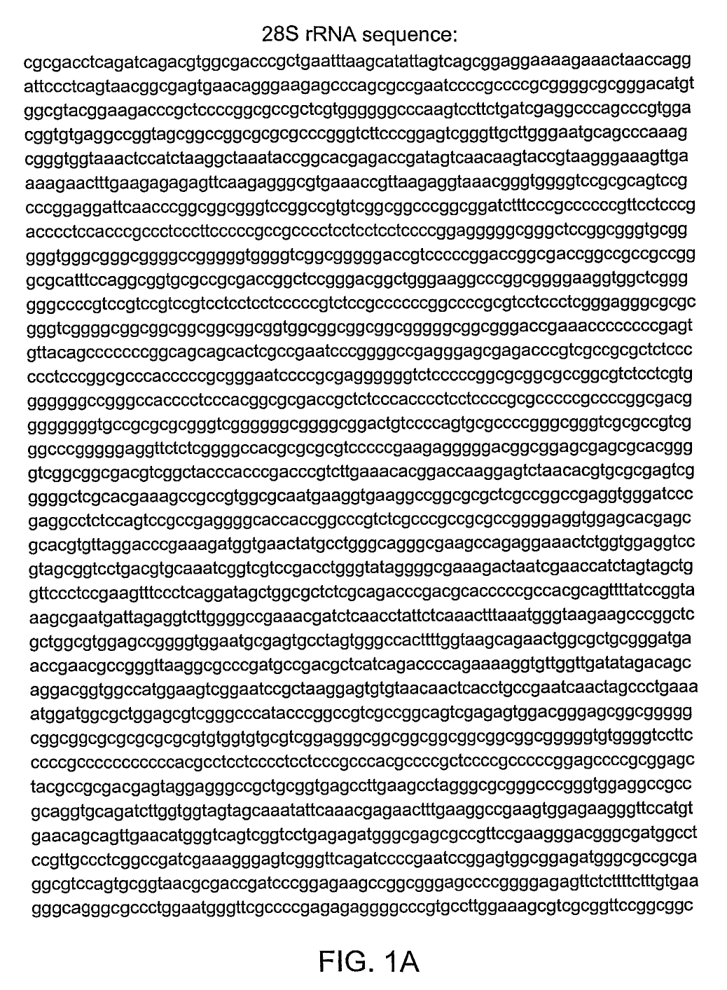

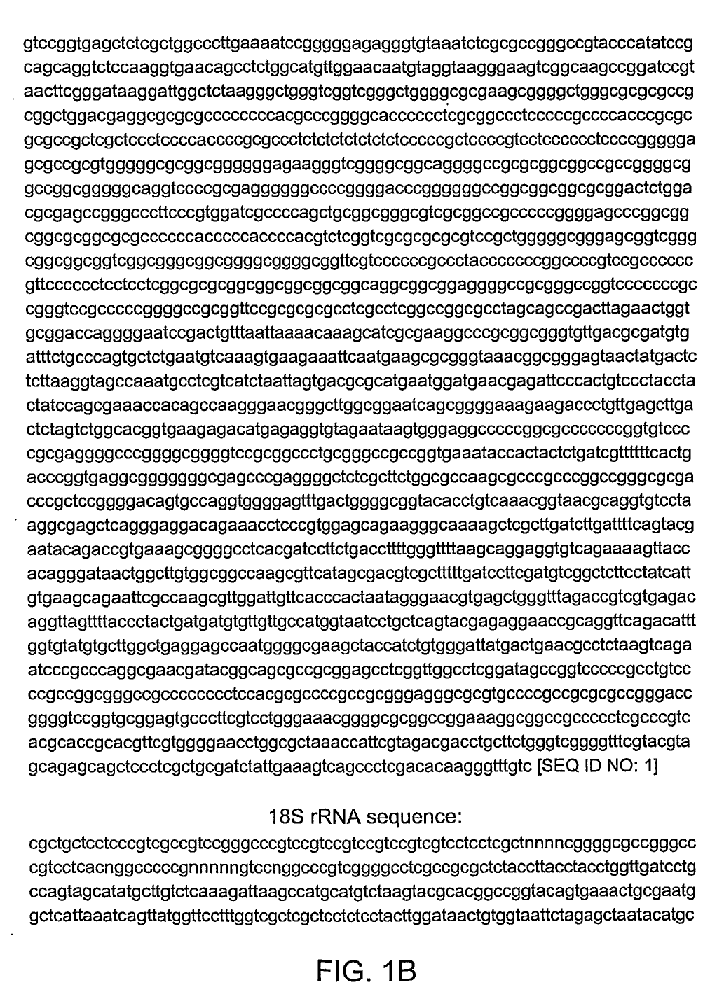

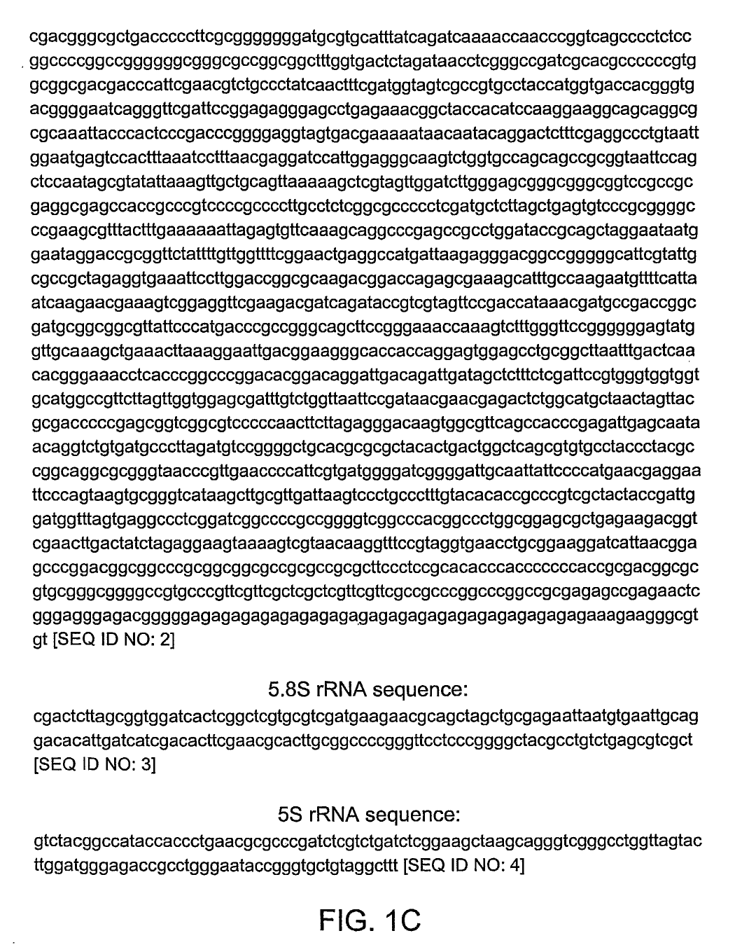

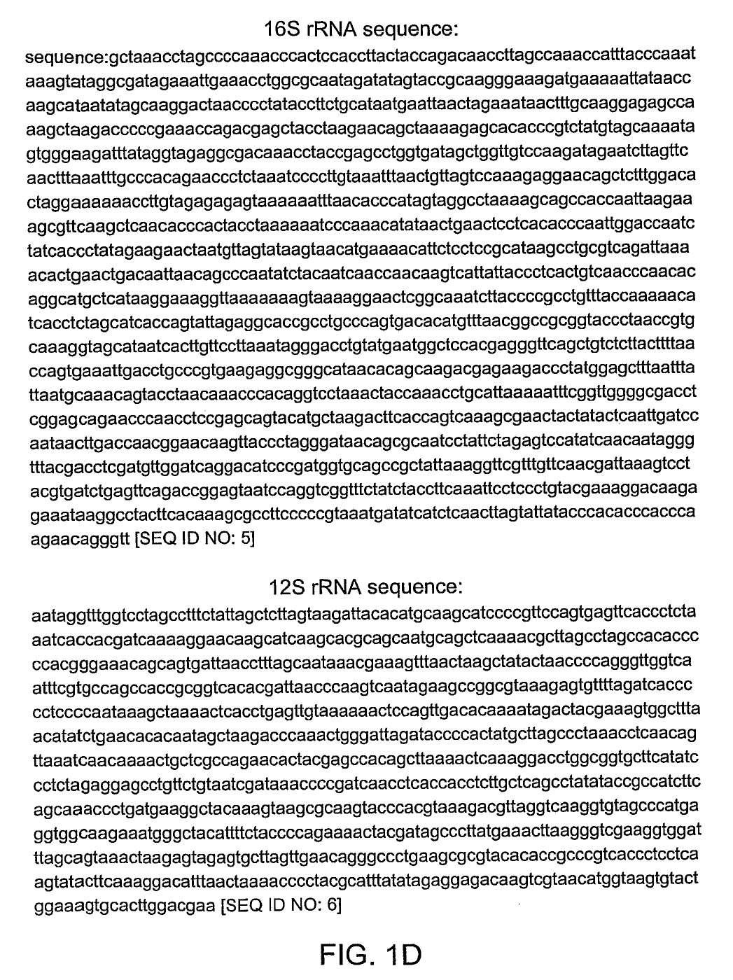

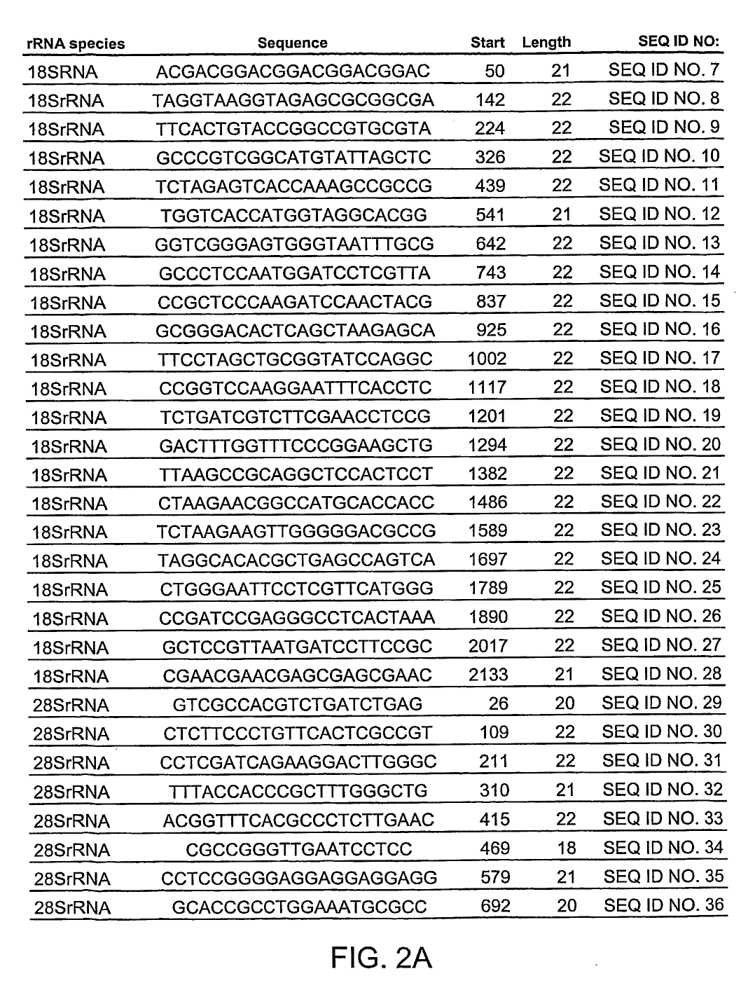

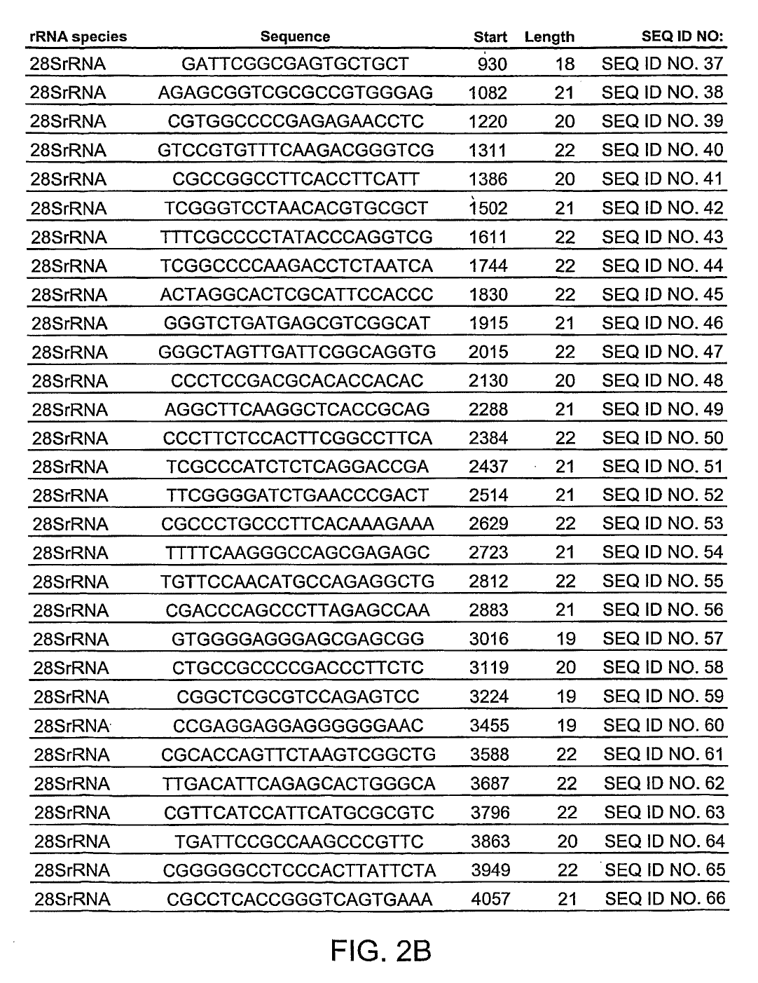

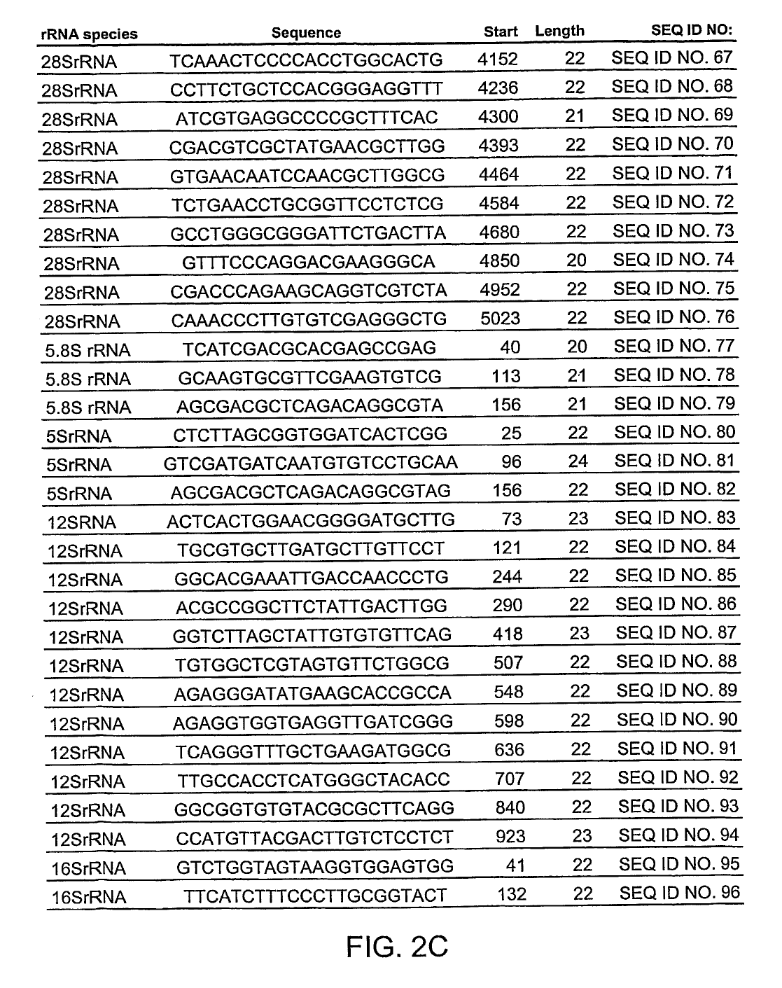

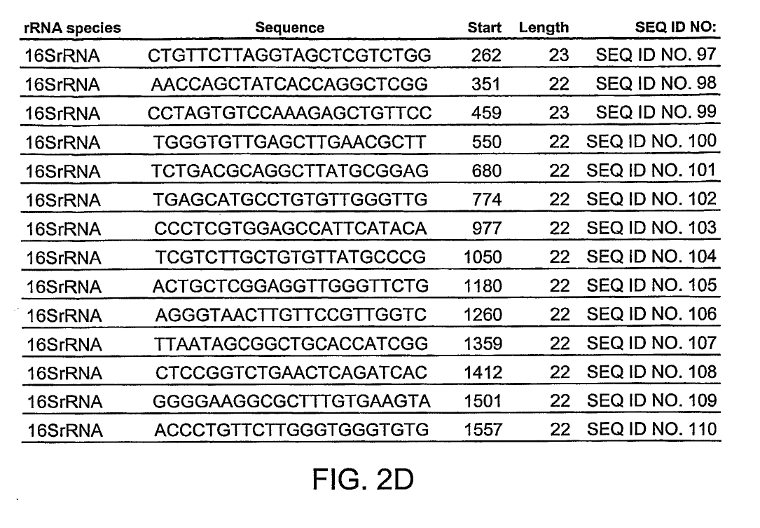

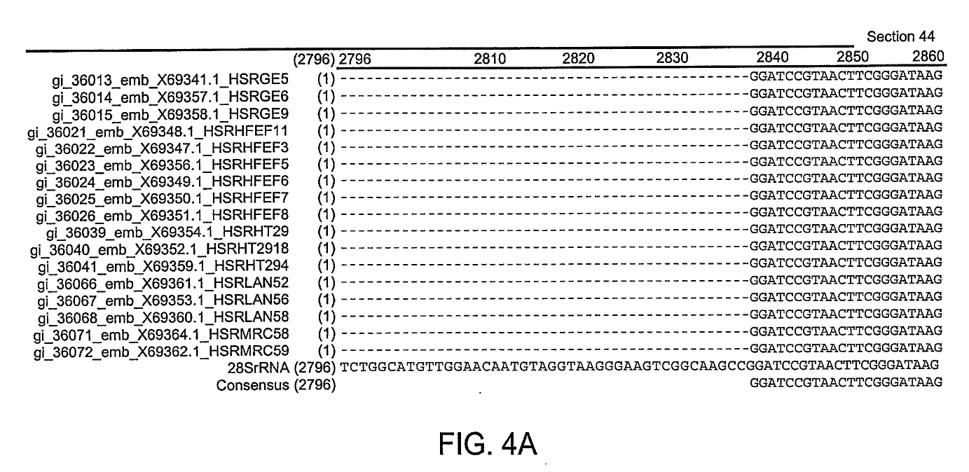

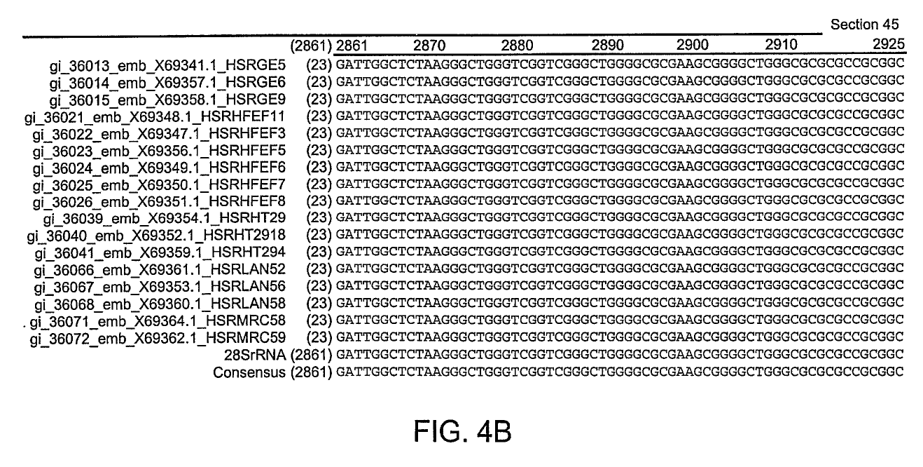

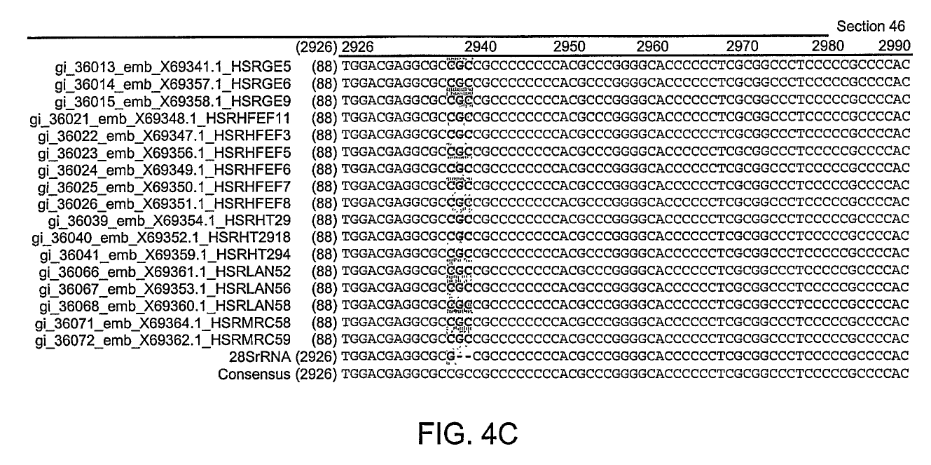

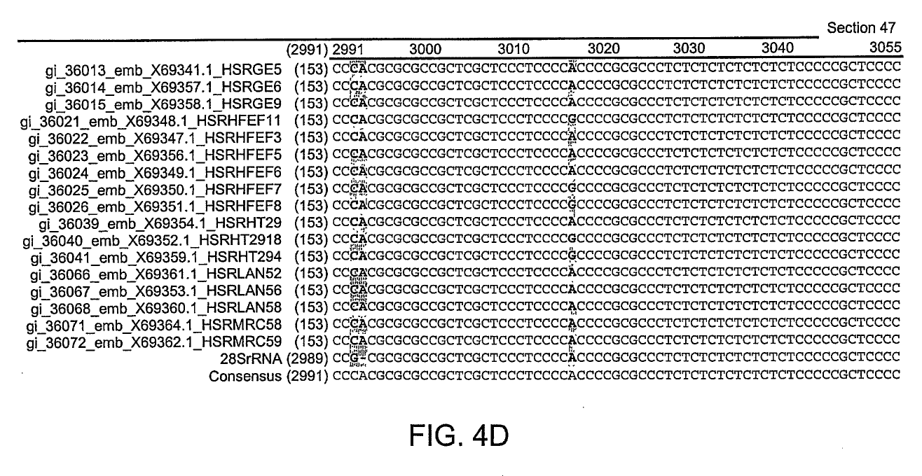

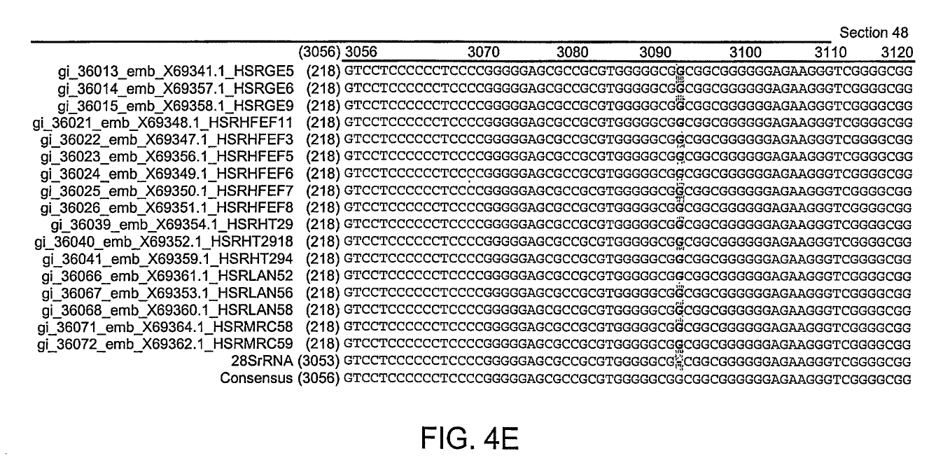

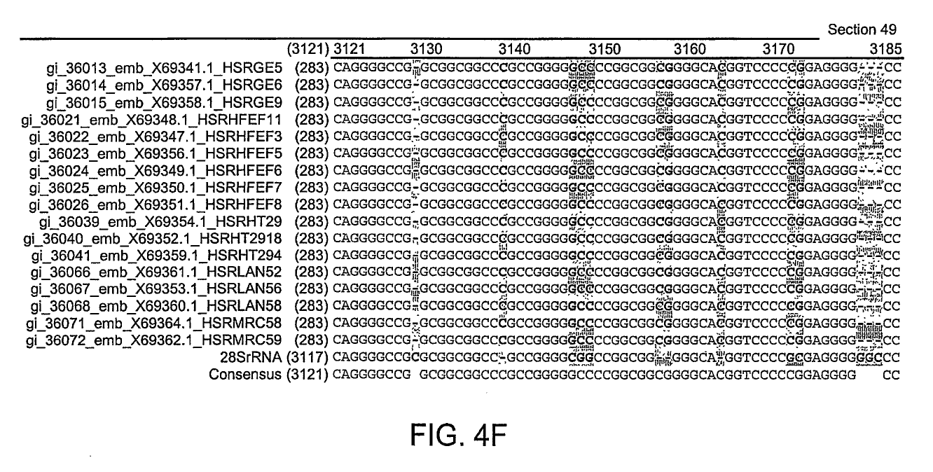

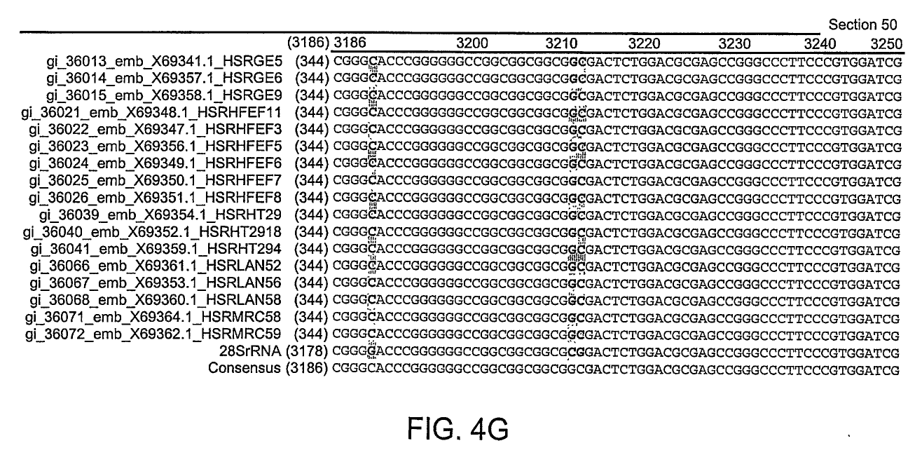

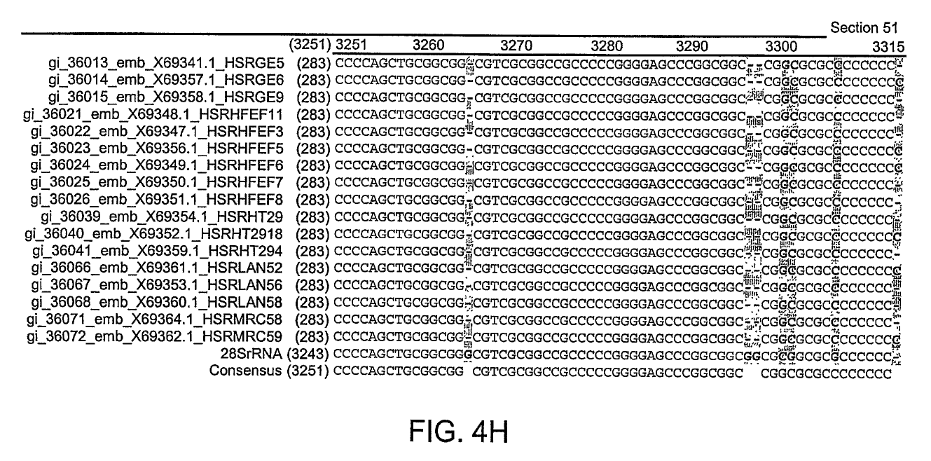

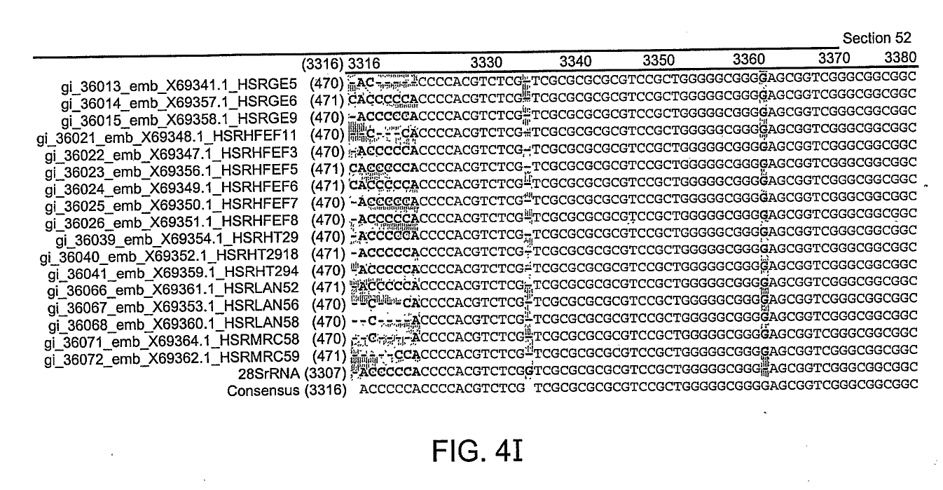

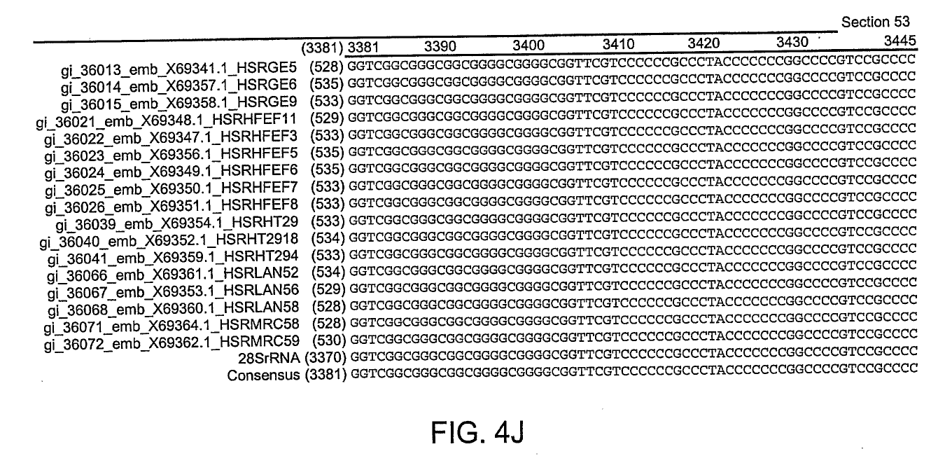

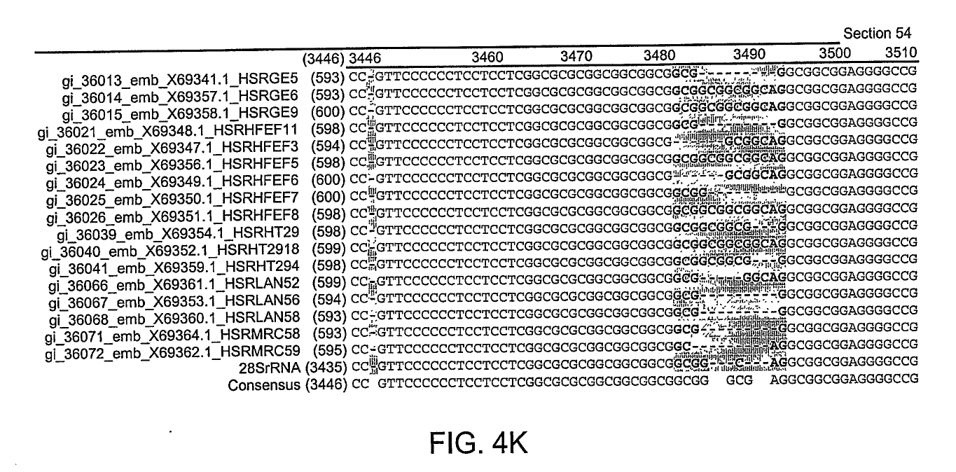

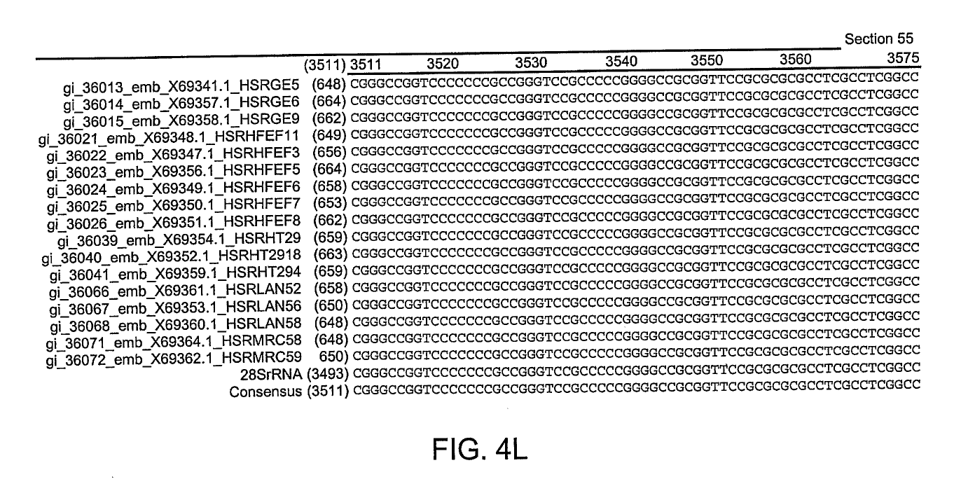

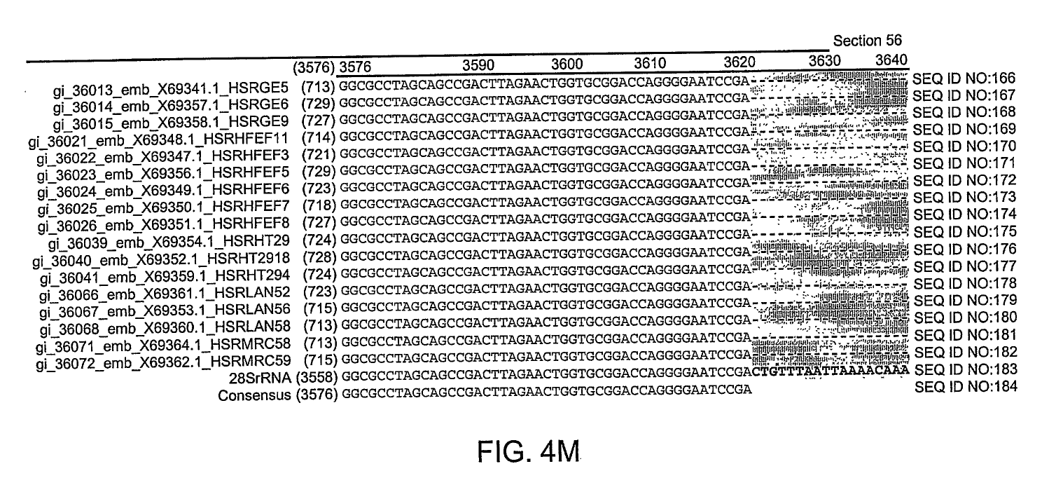

The rRNA and the rRNA fragments in the sample can comprise eukaryotic rRNA and eukaryotic rRNA fragments, although a sample also can comprise bacterial RNA species. In addition, the probes, including any tagged oligonucleotides, tagged single-stranded nucleic acid molecules, and/or DNA mimics, can be complementary to 18S, 28S, 5S, 5.8S, 12S, or 16S rRNA (see FIGS. 1A-1D and 2A-2D). The sequences of different rRNA species are highly conserved; however, the probes (e.g., oligonucleotides or molecules complementary to the rRNA species) can contain sequences complementary to polymorphic regions within the rRNA sequences to maximize removal of rRNA species from the sample (see, e.g., FIGS. 4A-4M for examples of such polymorphic regions). Such probes (e.g., oligonucleotides and/or molecules complementary to the rRNA species) can be used in various combinations. The rRNA and rRNA fragments can comprise at least two rRNAs selected from the group consisting of 28S, 18S, 5.8S, 5S, 12S, and 16S rRNAs. Thus, the plurality of probes can comprise one that is complementary to 18S rRNA and another that is complementary to 28S rRNA. Under certain circumstances, such as when an nrRNA of interest is present in low copy number, it can be desirable to remove unwanted RNA fragments that are abundant in the sample. It may be desirable to use an additional oligonucleotide and/or single-stranded nucleic acid molecule probe in addition to probes that are complementary to 18S and 28S, such as one or more or even all of 12S, 16S, 5.8S, and 5S rRNAs, alone or in further combination with probes (such as an oligonucleotide, a single-stranded nucleic molecule and/or a DNA mimic) that are complementary to tRNA and/or that are complementary to any mRNA that may be abundantly expressed in the RNA sample under investigation and that is not of interest.

Probes (e.g., oligonucleotide probes) can be synthesized using any in vitro chemical synthesis known in the art. Oligonucleotide probes can be as short as around 12 nucleotides or as long as around 150 nucleotides, such as around 15 or 17 nucleotides up to around 100 nucleotides, whereas a single-stranded nucleic acid molecule probe can be as short as 75 nucleotides up to the full-length of the RNA molecule that it complements. Oligonucleotides can be selected using a Primer selection program (e.g., Vector NTI (Invitrogen, Carlsbad Calif.) or Primer 3 (Rozen et al., “Primer 3 on the WWW for general users and for biologist programmers.” In: Krawetz and Misener, eds. Bioinformatics Methods and Protocols, Methods in Molecular Biology. Humana Press, Totowa, N.J. (2000), pp. 365-386)) using suitable parameters for primer length, base composition, and melting temperatures. It is preferred that the oligonucleotide probe have a GC content of about 35-80%, and a melting temperature of about 50-75° C. The optimal oligonucleotide length and melting temperature for the probes can be determined by the composition of the buffers used for the hybridization. Primer design programs select oligonucleotides based on the user's input of relevant parameters, including minimizing secondary structures, palindromic sequences, and hairpin loops. The oligonucleotides can be further analyzed by sequence homology search programs (e.g., BLAST) to minimize cross-hybridization to other genomic sequences, especially homology to expressed transcripts. It is preferred that the set of oligonucleotides used as probes for the hybridization contain at least one oligonucleotide complementary to each fragment (i.e., contiguous region) of the rRNA, wherein each fragment (i.e., contiguous region) is from about 100 base pairs to about 250 base pairs.

Single-stranded nucleic acid molecules can be synthesized in vitro (i.e., PCR or transcription-coupled amplification) or in vivo (i.e., replicated in living cells using recombinant DNA vectors). Such molecules can be synthesized as fragments or full-length molecules, and can be employed as probes in the context of the invention.

The probes of the invention can be tagged. By “tagged” is meant adding a molecule (a “tag”) to the probe (e.g., oligonucleotide, single-stranded nucleic acid molecule, and/or DNA mimic) that will permit downstream manipulations, such as removal. For example, the tag can be biotin, which, upon binding to streptavidin, such as streptavidin attached to a support (e.g., a magnetic bead or plastic surface), results in the removal of rRNA and any other RNA that is not of interest from the sample. Alternatively, the tag can be digoxygenin, which, upon binding to an immobilized anti-digoxygenin antibody, such as an antibody attached to a substrate (e.g., a magnetic bead or a microtiter plate), results in the removal of rRNA and any other RNA that is not of interest from the sample. Alternatively, the tag can be an amine linker that can be used to immobilize the oligonucleotide on a solid support.

The method of the invention comprises hybridizing the probes to the rRNA and rRNA fragments to form rRNA-probe complexes and rRNA fragment-probe complexes, which are then separated from the sample. Hybridization refers to the annealing of single stranded oligonucleotides to form double stranded oligonucleotides in an environment below the melting temperature of the double stranded oligonucleotide. Hybridization conditions are well known in the art and can be adapted to accommodate specific probes, rRNA sequences, and desired level of specificity of unwanted RNA removal. In this regard, “stringency” of hybridization reactions is determined by probe length, homology between the sequences, washing temperature, and salt concentration. In general, longer probes require higher temperatures for proper annealing, while shorter probes need lower temperatures. In addition, the higher the degree of homology between the probe and rRNA or rRNA fragment, the higher the relative temperature that can be used. Hybridization reactions are further described in, for example, Ausubel et al., Current Protocols in Molecular Biology, Wiley Interscience Publishers, (1995), and U.S. Pat. No. 7,081,340.

The hybridization conditions can include, but are not limited to, “stringent conditions” or “high stringency conditions” which typically: (1) employ low ionic strength and high temperature for washing, for example 0.015 M sodium chloride/0.0015 M sodium citrate/0.1% sodium dodecyl sulfate at 50° C.; (2) employ, during hybridization, a denaturing agent, such as formamide (e.g., 50% (v/v) formamide), 50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, optionally followed by a high-stringency wash consisting of 0.1×SSC containing EDTA at 55° C.

“Moderately stringent conditions” are described in, e.g., Sambrook et al., Molecular Cloning: A Laboratory Manual, New York: Cold Spring Harbor Press, 1989. Moderately stringent conditions can be achieved using a washing solution and hybridization conditions (e.g., temperature and ionic strength) less stringent that those described above. For example, moderately stringent conditions can comprise an overnight incubation at 37° C. in a solution comprising: 20% formamide, 5×SSC (150 mM NaCl, 15 mM trisodium citrate), 50 mM sodium phosphate (pH 7.6). The skilled artisan will recognize how to adjust the temperature, ionic strength, etc. as necessary to accommodate factors such as probe length and the like. DNA recovered in the remaining wash buffer can be added to the original hybridization mix, and hybridization can be repeated to further reduce the amount of rRNA in the sample. If desired, the rRNA of the sample is discarded, and the nrRNA is recovered in the hybridization buffer. In this regard, the hybridization step of the inventive method can be repeated to reduce the concentration of unwanted RNA, such as unwanted rRNA or rRNA fragments, in the sample. The hybridization step can be repeated by administering fresh probes to the reaction to form additional rRNA-probe complexes and rRNA fragment-probe complexes with the residual rRNA and rRNA fragments that may remain in the sample. Further reducing the concentration of rRNA and rRNA fragments further enriches the composition with desired nrRNA.

In view of the above, the invention further provides a composition comprising mRNA, which has been separated from a sample of total RNA and which includes N-terminal and/or internal molecules of mRNA. “N-terminal molecule of mRNA” means a molecule of mRNA that includes the transcription start site. “Internal molecule of mRNA” means a molecule of mRNA that does not include the transcription start site or the polyA tail. The mRNA can be amplified.

Also provided by the invention is an improved method of amplifying nrRNA fragments (such as mRNA or fragments thereof). The method comprises (i) separating nrRNA fragments from rRNA and rRNA fragments according to the inventive method described herein, and (ii) amplifying the nrRNA fragments. The improvement provided by the invention comprises using either of (i) nrRNA that has been separated from a sample of total RNA in accordance with the above-described method or (ii) the above-described composition. Although the inventive method enriches a sample for desired nrRNA, the composition can be further enriched with desired nrRNA by amplifying the nrRNA fragments in the resulting composition. The amplification of RNA, and oligonucleotides in general, has been the subject of numerous patents (see, e.g., U.S. Pat. Nos. 5,545,522; 5,716,785; 6,291,170; 6,642,034; 6,593,086; and 6,794,141, and U.S. Patent Application Publication No. 2005/0009101). Examples of methods that can be used to amplify mRNA include, but are not limited to, concatenation of RNA fragments, reverse transcription to generate the first cDNA strand, and strand displacement amplification; and attachment of an RNA polymerase promoter to the first or second strand cDNA followed by a transcription reaction to generate a multitude of copies of the starting cDNAs. A promoter is a sequence that can be recognized and bound by an RNA polymerase with subsequent transcription of the DNA fragment that is located downstream of the 3′ end of the promoter. Examples of such promoters include, but are not limited to, the T3, T4, T7, SP6, and Q beta replicase promoters.

The invention further provides an improved method of performing expression analysis of nrRNA. The method of analyzing nrRNA expression comprises (i) separating nrRNA fragments from rRNA and rRNA fragments according to the inventive method described herein and (ii) analyzing nrRNA expression. Examples of methods that can be used to perform expression analysis include, but are not limited to, PCR, quantitative PCR, and microarrays (PCR protocols, Bartlett et al., eds. Humana Press, Totowa, N.J. (2003); RT-PCR protocols, O'Connell, ed. Humana Press (2002); Gene cloning and analysis by RT-PCR, Siebert et al., eds. Eaton Publishing, Natick, Mass. (1998); A-Z quantitative PCR, Bustin, ed. IUL press, LaJolla, Calif. (2004); DNA microarrays: Gene expression applications, Jordan, ed. Springer-Verlag, New York, N.Y. (2001); Microarrays Methods and Applications: Nuts and Bolts, Hardiman, ed. DNA Press, Eagleville, Pa. (2003); and Guide to analysis of microarray data, 2^{nd} ed., Knudsen, ed. Wiley & Sons, Indianapolis, Ind. (2004)). The improvement provided by the invention comprises using either of (i) mRNA that has been separated from a sample of total RNA in accordance with the above-described method or (ii) the above-described composition.

A kit is also provided by the invention. The kit comprises instructions and at least one reagent for separating nrRNA from a sample of total RNA by (i) removing rRNA from the sample of total RNA, and, optionally, simultaneously or subsequently removing any other RNA that is not of interest, and (ii) purifying the nrRNA. The at least one reagent can comprise at least one oligonucleotide, single-stranded nucleic acid molecule, or DNA mimic that is complementary to (i) an rRNA selected from the group consisting of 18S, 28S, 12S, 16S, 5.8S and 5S and/or (ii) a tRNA.

In addition, the invention provides a kit for separating nrRNA fragments from a sample comprising rRNA and rRNA fragments (e.g., eukaryotic rRNA and eukaryotic rRNA fragments). Preferably, the rRNA and rRNA fragments comprise 28 S, 18 S, 5.8 S, 5S, 16S, and 12S rRNAs or fragments thereof. The inventive kit comprises a plurality of probes that hybridize to different rRNA target sequences of at least 50% of contiguous regions of about 100 base pairs of the rRNA, which contiguous regions comprise the rRNA targeting sequences. The plurality of probes also hybridize to rRNA fragments comprising the rRNA targeting sequences hybridized to by the probes in the contiguous regions of the rRNA. The kit also, optionally, further comprises instructions.

To increase specificity and efficiency of removal of unwanted rRNA from the sample, the probes of the kit preferably hybridize to rRNA targeting sequences of at least 60% of the contiguous regions (more preferably at least 70% of the contiguous regions). More preferably, the probes hybridize to rRNA targeting sequences of at least 80% of the contiguous regions (e.g., at least 90% of the contiguous regions). Even more preferably, the probes hybridize to rRNA targeting sequences of at least 95% of the contiguous regions (e.g., the probes hybridize to rRNA targeting sequences of all of the contiguous regions).

Also provided is a method of amplifying nrRNA fragments, such as those present in degraded RNA. The method comprises (i) separating an nrRNA fragment from rRNA and rRNA fragments according to the inventive method. The method further comprises (ii) ligating a first oligonucleotide to the 5′ end of nrRNA fragments, which have been separated from a sample (e.g., a sample of total RNA) in accordance with the above method. The first oligonucleotide (a) can comprise a first unique sequence, (b) preferably is of sufficient length to hybridize to a complementary oligonucleotide under annealing (i.e., hybridization) conditions as are known in the art, and (c) optionally contains an RNA polymerase promoter sequence. The first oligonucleotides serves as an anchor sequence at the 5′ end of nrRNA fragments, thereby facilitating the subsequent analysis of the fragments, in particular short fragments, such as those of less than about 50 base pairs. Desirably, the first oligonucleotide does not contain a sequence that is present in the genome of the species from which the nrRNA sample is derived. Preferably, the first oligonucleotide is at least about 10 nucleotides in length, more preferably, at least about 25 nucleotides in length, and most preferably, at least about 35 nucleotides in length. Examples of RNA polymerase promoter sequences include, but are not limited to, T3, T7, SP6, T4, and Qβ replicase. If desired, non-ligated nucleotides can be removed from the reaction.

The method further comprises (iii) annealing (i.e., hybridizing) to the nrRNA fragment a second oligonucleotide. The second oligonucleotide preferably comprises a second unique sequence and a random sequence, wherein the random sequence is of sufficient length to hybridize to the nrRNA under annealing (i.e. hybridization) conditions as are understood in the art. Desirably, the unique sequence of the second oligonucleotide does not contain a sequence that is present in the genome of the species from which the nrRNA sample is derived. Optionally, the unique sequence of the second oligonucleotide comprises an RNA polymerase promoter. As such, it is possible to obtain a nucleotide chain comprising RNA polymerase promoters capable of directing transcription in opposite directions. The random sequence can be from about 6 to about 35 nucleotides in length, preferably from about 6 to about 12 nucleotides in length. The random sequence must anneal to the nrRNA to be amplified. One of ordinary skill in the art will appreciate that step (iii) involves heating the sample and then cooling the sample to allow the second oligonucleotide to anneal to the nrRNA (e.g., mRNA) and incubating the sample at the annealing temperature in accordance with methods known in the art.

The method still further comprises (iv) extending a DNA strand from the second oligonucleotide to generate a first cDNA having a 5′ and a 3′ end. The DNA strand can be extended using any suitable method such as, for example, reverse transcription. Several methods of reverse-transcription for obtaining cDNA are known in the art. If the sample of nrRNA is small, it may be desirable to amplify the cDNA. Therefore, if desired, the cDNA may be amplified using any suitable method, such as PCR. Then, the method comprises (v) degrading the nrRNA (i.e., the nrRNA fragment). The nrRNA can be degraded using one or more RNA-degrading enzymes, such as RNases. The enzymes will need to be removed prior to transcription, such as by phenol-chloroform extraction in accordance with methods known in the art. The method optionally further comprises removing any free oligonucleotides and/or RNA fragments in accordance with methods known in the art, such as size-exclusion column purification, preferential precipitation, and the like.

Afterwards, the method comprises (vi) synthesizing the complementary strand to the first cDNA by annealing a third oligonucleotide to the 3′ of the first cDNA and extending a strand from the third oligonucleotide. In other words, a complementary strand to the first cDNA is synthesized by (a) contacting the first cDNA with a third oligonucleotide such that the third oligonucleotide hybridizes to the 3′ end of the first cDNA, and (b) extending the complementary strand from the third oligonucleotide, whereupon a double-stranded DNA copy of the nrRNA fragment is generated. The third oligonucleotide is of sufficient length to hybridize to at least a portion of the first oligonucleotide under annealing conditions and optionally contains an RNA polymerase promoter sequence as previously described. The third oligonucleotide can comprise a unique sequence that is not present in the genome of the species from which the nrRNA sample was extracted. If the free oligonucleotides and/or RNA fragments are not removed in step (v), then step (vi) should be conducted at an elevated annealing temperature to prevent the second oligonucleotide and any RNA fragments from annealing to the first cDNA, in which case a thermostable DNA polymerase, preferably with proofreading capabilities, is used for synthesis of the complementary strand to the first cDNA.

The method optionally comprises (vii) purifying the double-stranded DNA copy of the nrRNA (i.e., the nrRNA fragment) in accordance with methods known in the art (e.g., binding to glass milk or ion-exchange resin, proteinase K digestion and phenol-chloroform extraction, etc.). In addition, the double-stranded DNA copy can be amplified using, for instance, PCR. The method further comprises (viii) transcribing RNA from the double-stranded DNA in accordance with methods known in the art, such as with commercially available kits (e.g., such as those available from Ambion (Austin, Tex.) or Epicentre (Madison, Wis.)). The purified double-stranded copy of the nrRNA carries two RNA polymerase promoters that can be used to generate RNA copies of the coding or the noncoding strands.

In addition, the invention provides a method of amplifying a cDNA complementary to an nrRNA fragment. The method comprises (i) separating an nrRNA fragment from rRNA and rRNA fragments according to the invention. The method further comprises (ii) ligating a first oligonucleotide, such as the first oligonucleotide described above, to the 5′ end of the nrRNA fragment. The method then comprises (iii) hybridizing to the nrRNA fragment a second oligonucleotide and (iv) extending a DNA strand from the second oligonucleotide to generate a first cDNA. The first cDNA is then (v) amplified, thereby amplifying a cDNA complementary to an nrRNA fragment.

In view of the above, the invention provides a kit comprising instructions and at least one reagent for generating a double-stranded DNA copy of mRNA in accordance with the above-described kit. The kit can further comprise the kit described above, in which case the at least one reagent comprises at least one tagged oligonucleotide, single-stranded nucleic acid molecule, or DNA mimic that is complementary to (i) an rRNA selected from the group consisting of 18S, 28S, 12S, 16S, 5.8S and 5S and/or (ii) a tRNA.

The invention further provides a method of determining the level of nrRNA in a sample of nrRNA, which has been separated from total RNA as described above. The method comprises (i) separating nrRNA fragments from rRNA and rRNA fragments according to the inventive method, and (ii) labeling the separated nrRNA with a detectable label. The nrRNA can be labeled by any suitable method as is known in the art. Examples of suitable labels include biotin, fluorescent tags, radioactive labels, and the like. The method further comprises (iii) providing an array comprising a collection of fixed DNAs that hybridize to a plurality of target sequences located within an expression sequence of a gene of interest. The fixed DNAs (e.g., DNA fragments) comprise from about 15 nucleotides to about 750 nucleotides, such as from about 30 nucleotides to about 750 nucleotides. At least one target sequence is interspersed approximately every 500 base pairs of the expression sequence. In other words, preferably, a fixed DNA hybridizes to a target sequence that is within 500 base pairs of another target sequence. More preferably, a target sequence recognized by a fixed DNA is located less than 500 base pairs from other target sequences recognized by fixed DNAs. Also preferably, the collection of fixed DNAs comprises a sufficient number of fixed DNAs to hybridize to target sequences of about 50% or more of the expression sequence. In other words, the collection preferably comprises DNA fragments that correspond to at least about 50% of each gene of interest and, preferably, the collection comprises at least one unique or discrete DNA fragment for every 500 contiguous base pairs of each gene of interest. The method further comprises (iv) applying the labeled nrRNA fragments to the array under hybridization conditions, i.e., under conditions that allow annealing of the labeled nrRNA with complementary fragments in the array of DNA fragments, and (v) optionally removing unbound and nonspecifically bound nrRNA fragments from the array. The method then comprises (vi) detecting the labeled nrRNA fragments that have specifically annealed to the fixed DNAs in the array (i.e., the nrRNA that has annealed to complementary fragments in the array of DNA fragments); and (vii) quantitating the level of labeled nrRNA fragments detected in (iv) to thereby determine the level of nrRNA in the sample. The method can comprise (viii) correlating the level of nrRNA in the sample to a level of expression of a gene of interest. For example, the amount of nrRNA in the sample indicates whether the gene of interest is highly or poorly expressed.

An array for use in the inventive method can include any one-dimensional, two-dimensional or substantially two-dimensional (as well as a three-dimensional) arrangement of addressable regions bearing particular probes associated with that region. The arrays for use in the invention generally are arrays of probes as described above, and can include nucleic acids, including oligonucleotides, polynucleotides, cDNAs, RNAs, synthetic mimetics thereof, and the like. Oligonucleotide probes can be attached to the array substrate at any point along the nucleic acid chain, but are generally attached at one of their termini (e.g., the 3′ or 5′ terminus).

Any suitable array substrate can be used in the context of the invention including, for example, siliceous materials, e.g., glass, fused silica, ceramics and the like; metals such as stainless steel, aluminum, and alloys thereof; polymers, e.g., plastics and other polymeric materials such as polysulfone, poly(vinylidene fluoride), poly(ethyleneterephthalate), polyurethane, e.g., nonporous polyurethane, fluoropolymers such as polytetrafluoroethylene (e.g., Teflon™) or the like, polypropylene, polystyrene, polycarbonate, PVC, nylon, and blends thereof. With arrays that read using fluorescence, the substrate may be of a material that emits low fluorescence upon illumination with the excitation light. Additionally, the substrate may be relatively transparent to reduce the absorption of the incident illuminating laser light and subsequent heating if the focused laser beam travels too slowly over a region. Any given substrate may carry one, two, four or more arrays disposed on a front surface of the substrate. Depending upon the use, multiple arrays can be used which are the same or different from one another. Each array may cover an area of less than 100 cm², or even less than 50 cm², 10 cm² or 1 cm². The substrate carrying one or more arrays can be any shape, such as a rectangular solid (although other shapes are possible). Arrays are further described in, for example, U.S. Pat. No. 7,022,157.

Arrays can be fabricated in a number of ways, including, for example, drop deposition, direct synthesis, light directed fabrication, or by printing. Methods of generating arrays are further described in, for example, U.S. Pat. Nos. 6,171,797; 6,180,351; 6,232,072; 6,242,266; and 6,323,043, as well as U.S. patent application Ser. No. 09/302,898, filed Apr. 30, 1999 by Caren et al. (a counterpart of which has been published as U.S. Patent Application No. 2004/0203138 A1), and the references cited therein, which are hereby incorporated by reference. For example, the labeled nrRNA can be incubated with an array as described herein in a commercially available buffer (e.g., such as those available from Sigma Chemical Co., St. Louis, Mo.), for example.

Unbound and nonspecifically bound nrRNA can be removed by washing in a salt-containing buffer at various temperatures/stringencies. The method employed in detecting the labeled nrRNA will be determined largely by the label employed in accordance with well-known methods. The level of detected labeled nrRNA can be quantitated after subtracting the background. If desired, the nrRNA can be initially amplified in accordance with the above-described methods.

Accordingly, in view of the above, the invention also provides a microarray for analysis of nrRNA fragments for genes of interest. The microarray comprises a collection of DNA fragments, wherein each fragment ranges in size from about 15 nucleotides to about 750 nucleotides (e.g., from about 30 base pairs to about 750 base pairs), such as from about 50 base pairs to about 600 base pairs or from about 60 base pairs to about 500 base pairs. The collection preferably comprises at least one unique DNA fragment (or discrete DNA fragment) for every 500 contiguous base pairs of each gene of interest. In other words, the collection preferably comprises a DNA fragment (i.e., probe) that hybridizes to a target DNA (e.g., gene) of interest within 500 base pairs of another DNA fragment of the collection. Preferably, the collection comprises DNA fragments that correspond to at least about 50% of each gene of interest, as described further herein.

Thus, further provided is a kit comprising instructions and at least one reagent for separating nrRNA from a sample of total RNA by (i) removing rRNA from the sample of total RNA, and, optionally, simultaneously or subsequently removing any other RNA that is not of interest, and (ii) purifying the nrRNA, and the above-described microarray. The at least one reagent comprises at least one tagged oligonucleotide, single-stranded nucleic acid molecule, or DNA mimic that is complementary to (i) an rRNA selected from the group consisting of 18S, 28S, 12S, 16S, 5.8S and 5S and/or (ii) a tRNA.

In addition, the invention provides a system for separating nrRNA fragments from a sample comprising rRNA and rRNA fragments, such as eukaryotic rRNA (e.g., comprise 28S, 18S, 5.8S, 5S, 16S, and 12S rRNAs or fragments thereof). The system comprises (i) a device adapted for the separation of nucleic acids by hybridization; and (ii) a plurality of probes positioned within or upon the device to separate the rRNA and rRNA fragments from nrRNA. The probes hybridize to (a) different rRNA target sequences of at least 50% of contiguous regions of about 100 base pairs of the rRNA, which contiguous regions comprise the rRNA targeting sequences. Preferably, the probes of the plurality hybridize to rRNA targeting sequences of at least 60% of the contiguous regions (more preferably at least 70% of the contiguous regions) to further increase the efficiency of separation. More preferably, the probes hybridize to rRNA targeting sequences of at least 80% of the contiguous regions (e.g., at least 90% of the contiguous regions). Even more preferably, the probes hybridize to rRNA targeting sequences of at least 95% of the contiguous regions (e.g., the probes hybridize to rRNA targeting sequences of all of the contiguous regions). The probes also hybridize to (b) rRNA fragments comprising the rRNA targeting sequences hybridized to by the probes in the contiguous regions of the rRNA.

Any device that employs hybridization to separate of nucleic acids can be used in the context of the inventive method. For example, the device can be an array, such as the array described above, where probes are attached to a substrate to facilitate separating target oligonucleotides from a sample. The device can be a filtration apparatus wherein probes are attached to filters to allow oligonucleotide separation following centrifugation. Likewise, the device can be a microfluidic device. Microfluidic devices can comprise biochannels or microchannels comprising arrays of probes to capture target RNA in samples. Alternatively, a microfluidic reaction apparatus can be configured to have one or more individual reaction chambers in direct communication with a microarray of oligonucleotide probes to perform multiple, parallel, thermally controlled hybridization reactions. Microfluidic-style devices have been developed for oligonucleotide amplification, detection, and/or hybridization assays, as described in International Patent Applications WO 96/15450; WO 96/15576; WO 97/27324; WO 96/39252; WO 96/39260; WO 97/16561; WO 97/16835; WO 97/37755; WO 97/43629; and WO 98/13683; U.S. Pat. Nos. 5,061,336; 5,071531; 5,110,745; 5,126,022; 5,135,627; 5,147,607; 5,296,375; 5,304,487; 5,486,335; 5,498,392; 5,569,364; 5,585,069; 5,587,128; 5,593,838; 5,603,351; 5,631,337; 5,632,876; 5,637,469; 5,643,738; 5,681,484; 5,726,026; 5,747,169; 5,750,015; 5,755,942; 5,770,029; 5,843,767; and U.S. Patent Application Publication No. 2005/0009101 A1.

The following examples serve to illustrate the invention but are not intended to limit its scope in any way.

### EXAMPLE 1

This example describes a method of recovering mRNA from paraffin-embedded tissues by subtraction with oligonucleotides complementary to rRNA.

Five 10 micron sections of paraffin-embedded, benign prostatic hyperplasia tissues are extracted 3× with xylene to remove the paraffin and 3× with ethanol. After air drying, the tissue is incubated in 100 μl of proteinase K buffer at 50° C. for four days, with additional aliquots of proteinase K buffer added every 12 hours. The proteinase K buffer comprises 10 millimolar Tris (pH 8), 5 millimolar EDTA, 100 millimolar NaCl, 0.1% SDS, and 2 μg/mL proteinase K. The RNA is then isolated using total RNA isolation methods, such as lysis in guanidium thiocyanate followed by phenol-chloroform extraction and ethanol precipitation. A number of commercial kits are suitable for this purpose, such as Totally RNA (Ambion), Perfect RNA Eukaryotic Kit (Eppendorf, Westbury, N.Y.), and RNeasy kit (Qiagen, Valencia, Calif.). The RNA is extracted according to manufacturer's directions. Following extraction and precipitation, the RNA is subjected to DNase treatment, phenol chloroform extraction, and precipitation. The RNA resuspended in H₂O, and stored at −80° C.

To remove the rRNA, one microgram of the total RNA is incubated with biotinylated oligonucleotides in a hybridization buffer to promote the annealing of the oligonucleotide to the rRNA fragments. A sample of suitable oligonucleotides is shown in FIGS. 2a-2d. Any suitable buffer can be used for this step, including buffers containing tetramethylammionium chloride (TMACl; see, e.g., U.S. Pat. No. 5,633,134 in regard to the use of quaternary ammonium salt). One such buffer is composed of 50 mM Tris HCl, pH 8.0, 150 mM NaCl, 10 mM EDTA, and 0.1% SDS. The oligonucleotide mix includes an equimolar amount of all oligonucleotides that are complementary to 28S, 18S, 5.8S, 5S, 12S, and 16S rRNA. One microgram of RNA and 2 micrograms of oligonucleotides are resuspended in 10 μl of H₂O and incubated at 75° C. for 10 minutes, followed by immersion in an ice bath for 10 minutes. Ten microliters of 10× buffer and 80 μl of H₂O are added, and the solution is incubated at 65° C. for between 10 minutes and several hours. Following the incubation step, a 50 μl aliquot of streptavidin-coated magnetic beads (Dynal, Oslo, Norway) is added to the hybridization solution, which is then mixed gently to allow for binding of the biotinylated oligonucleotides to the strepavidin-coated beads. The beads are then precipitated using a magnet, and the supernatant is transferred to a fresh tube. If desired, the hybridization step can be repeated one or more times by adding a fresh aliquot of the oligonucleotides. The supernatant is extracted with phenol-chloroform, and the RNA is precipitated with the addition of 2 volumes of ethanol. The RNA is resuspended in H₂O, quantitated using the picogreen DNA quantitation kit (Invitrogen, Carlsbad, Calif.), and stored at −80° C. The RNA is then ready for reverse transcription.

This example illustrates a method of separating nrRNA from rRNA and rRNA fragments using the inventive method.

### EXAMPLE 2

This example demonstrates a method of preparing probes for use in the invention.

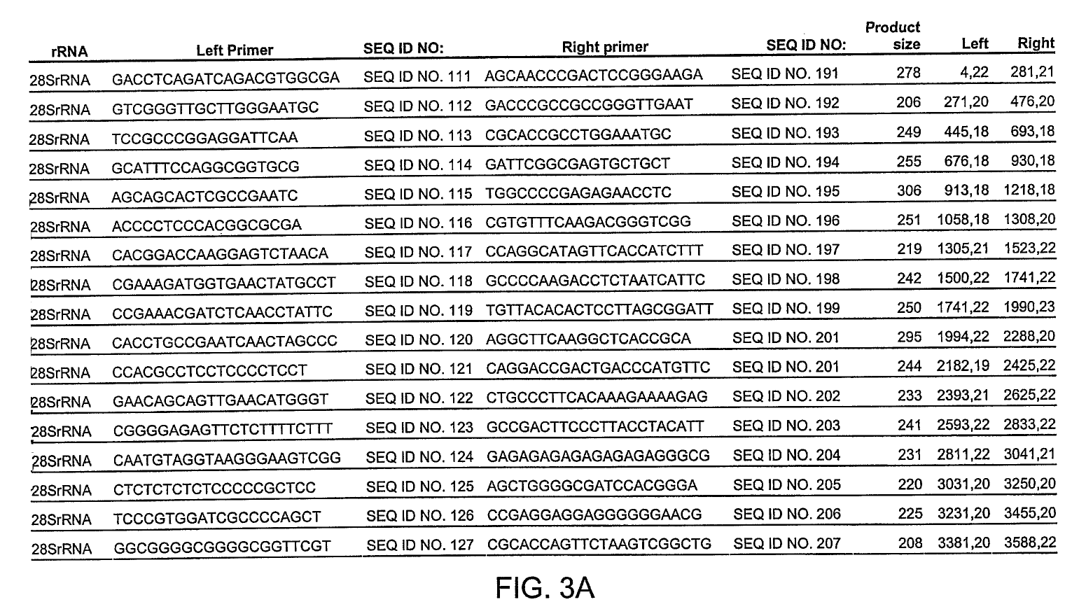

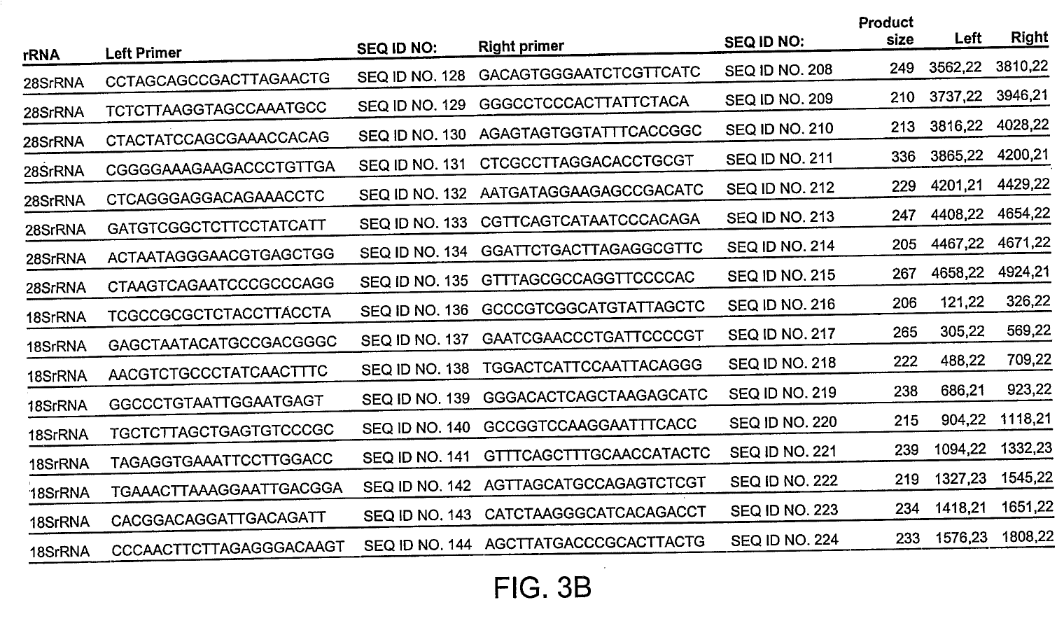

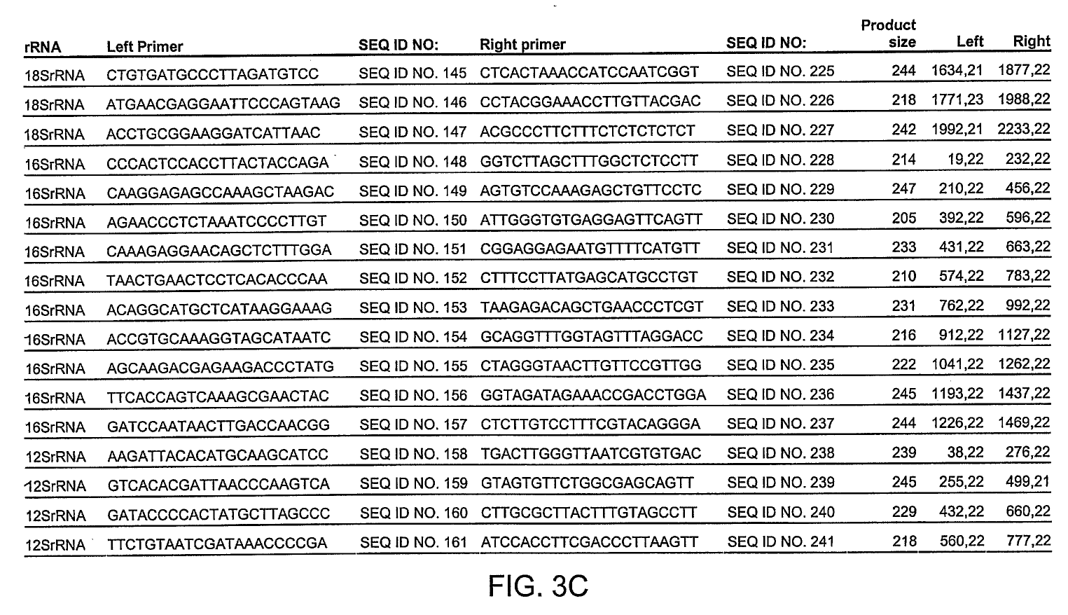

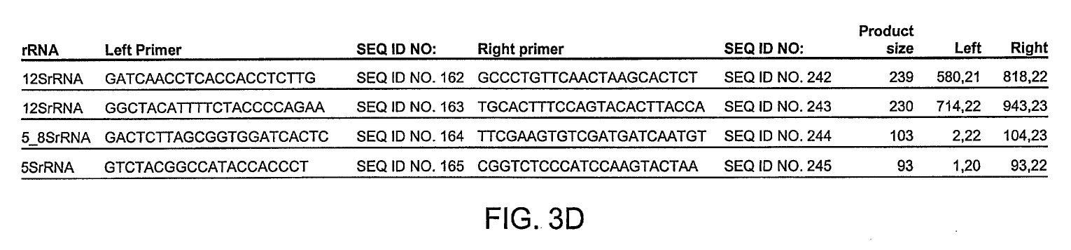

To generate single-stranded DNA fragments complementary to rRNA, PCR primers are designed to amplify segments of the rRNA species that range in size from around about 100 base pairs up to the full-length rRNA template. The primers listed in FIGS. 3A-3C are designed to amplify fragments corresponding to all six rRNA species. The amplification reaction is performed using MasterTaq kit from Eppendorf according to the manufacturer's direction. The buffer is supplemented with 10% DMSO or 5% formamide when needed to improve the amplification of GC rich sequences. The amplification is performed for a total of 35 cycles of denaturation at 94° C. for 30 seconds, annealing at 60° C. for 30 seconds, and extension for 1 minute at 72° C. in the presence of 25 pmoles of the forward and reverse primers. The amplified rRNA fragments are used as templates to generate the single-stranded DNA complementary to the rRNA using asymmetric PCR reactions. This is achieved by reducing the concentration of the forward primer to 2.5 pmoles during the PCR amplification, which leads to an excess of the desired strand. Following asymmetric PCR, the DNA is purified using phenol-chloroform extraction and ethanol precipitation. The DNA fragments are quantitated and mixed in equimolar amounts.

To remove rRNA from a sample comprising total RNA, 1 μg of the total RNA is incubated with 2 μg of the single-stranded DNA fragments complementary to rRNA. The hybridization and rRNA removal are performed as described above.

This example illustrates a method of preparing probes for use in the inventive method, kit, and system.

### EXAMPLE 3

This example describes a method of analyzing nrRNA expression. In particular, this example describes reverse transcription and detection of p53 mRNA.

Reverse transcription is performed using the cMaster RTₚₗᵤₛ PCR kit (Eppendorf) using random primers or a combination of random primers and oligo dT primers according to the supplier's directions. Twenty nanograms of selected RNA (e.g., nrRNA isolated according to the method of Example 1) are used for each reaction. The reaction is stopped by heating at 70° C. for 15 minutes, followed by the addition of 1 unit of Rnase H and incubation at 37° C. for 20 minutes to degrade the RNA. The cDNA is now ready for RT-PCR. The transcription reaction is stored at −20° C.

To detect the p53 transcript, 1 μl of the cDNA is amplified using primers designed to amplify a portion of the p53 transcript. Examples of suitable primer pairs are:

| | |
|---|---|
| F1: cttgccgtcccaagcaatggatg; | (SEQ ID NO: 185) |
| R1: ggagcttcatctggacctgggtc | (SEQ ID NO: 246) |
| (89 base pairs), | |
| F2: caataggtgtgcgtcagaagcacc; | (SEQ ID NO: 186) |
| R2: caaaacaccagtgcaggccaacttg | (SEQ ID NO: 247) |
| (86 base pairs), and | |
| F3: ggtctcacagtgttgcccaggctg; | (SEQ ID NO: 187) |
| R3: ttgtaatcccagcactctgggag | (SEQ ID NO: 248) |
| (86 base pairs). | |

One microliter of the cDNA is amplified using the primer pairs shown above for 40 cycles using the MasterTaq PCR amplification kit (Eppendorf) in a 15 μl reaction containing 25 pmoles of each primer. The annealing temperature is 60° C. The reactions are separated on a 7.5% acrylamide gel and stained with ethidium bromide to detect the amplification products.

This example illustrates a method of analyzing nrRNA expression from a sample of nrRNA separated from rRNA in accordance with the invention.

### EXAMPLE 4

This example describes amplification of mRNA fragments using transcription-coupled amplification.

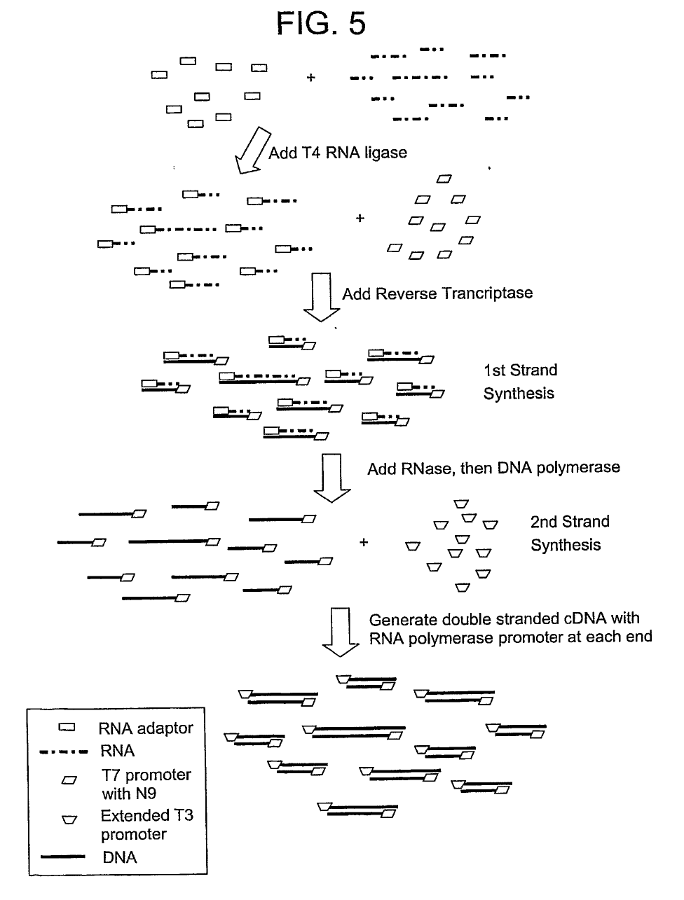

One hundred nanograms of selected RNA fragments (e.g., nrRNA fragments isolated according to the method of Example 1) are ligated to 1 picomole of RNA oligonucleotide (GGAUGAACGUAGGAAGCUUG (SEQ ID NO: 188)) using 2 units of T4 RNA ligase in a 20 μl-reaction (NEB, Beverly, Mass.) according to supplier's recommendations. Following the ligation reaction, the RNA is purified by adding 80 μl of TE8 to the ligation reaction and then gently pipetting the entire volume to the top of a Sephadex G25 column (Pharmacia, Peapack, N.J.). The reaction is spun at 4,000 rpm for 1 minute, and the recovered RNA is precipitated by adding 0.1 volumes of 5.2 M sodium acetate and 2 volumes of ethanol. The RNA is resuspended in 20 μl of TE8. The reverse transcription reaction is performed as described in the Superscript Choice System for cDNA synthesis (Invitrogen), except that the primers used to initiate the priming of the first strand are composed of a T7 promoter followed by around 6 to 12 random nucleotides (GGAATTAATACGACTCACTATAGGGN9 (SEQ ID NO: 189)). The reaction is treated with RNase H at 30° C. for 30 minutes. The second strand synthesis is initiated using the following primer, which includes a T3 promoter (GCGCGAAATTAACCCTCACTAAA GGGATGAACGTAGGAAGCTTG (SEQ ID NO: 190)). Following the second strand synthesis, the cDNA is extracted with phenol-chloroform, ethanol precipitated, and resuspended in TE8. The cDNA (FIG. 5) is ready for the amplification step using the MEGAshortscript high yield transcription kit (Ambion). The transcription reaction is performed according to the manufacturer's recommendations. Depending on the choice of promoter used for the transcription reaction, the sense or the antisense strand can be generated. The amplified RNA can serve as a template for reverse transcription and RT-PCR as described in Example 3 or as a probe for use in microarray hybridization.

This example illustrates a method of amplifying nrRNA separated from rRNA according to the method of the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of separating non-ribosomal transcribed RNA (nrRNA) fragments from ribosomal RNA (rRNA) and rRNA fragments, wherein the method comprises:
(i) providing a sample comprising rRNA, rRNA fragments, and nrRNA fragments, wherein the rRNA comprises multiple contiguous regions of about 100 base pairs, each contiguous region comprising an rRNA targeting sequence, and wherein the rRNA fragments comprise one or more of the rRNA targeting sequences;
(ii) providing a plurality of probes, wherein the probes hybridize to (a) different RNA targeting sequences of at least 50% of the contiguous regions of the rRNA and (b) rRNA fragments comprising the rRNA targeting sequences hybridized to by the probes in the contiguous regions of the rRNA;
(iii) adding the plurality of probes to the sample,
(iv) hybridizing the probes to the rRNA and rRNA fragments to form rRNA-probe complexes and rRNA fragment-probe complexes, and
(v) separating the rRNA-probe complexes and rRNA fragment-probe complexes from the sample,
thereby separating nrRNA fragments from rRNA and rRNA fragments.

2. The method of claim 1, wherein the method further comprises (vi) recovering and purifying the nrRNA fragments.

3. The method of claim 1, wherein the sample comprises total RNA.

4. The method of claim 1, wherein the probes hybridize to rRNA targeting sequences of at least 60% of the contiguous regions.

5. The method of claim 4, wherein the probes hybridize to rRNA targeting sequences of at least 70% of the contiguous regions.

6. The method of claim 5, wherein the probes hybridize to rRNA targeting sequences of at least 80% of the contiguous regions.

7. The method of claim 6, wherein the probes hybridize to rRNA targeting sequences of at least 90% of the contiguous regions.

8. The method of claim 7, wherein the probes hybridize to rRNA targeting sequences of at least 95% of the contiguous regions.

9. The method of claim 8, wherein the probes hybridize to rRNA targeting sequences of all of the contiguous regions.

10. The method of claim 1, wherein the rRNA and the rRNA fragments comprise eukaryotic rRNA and eukaryotic rRNA fragments.

11. The method of claim 1, wherein the rRNA and rRNA fragments comprise at least two rRNAs selected from the group consisting of 28S, 18S, 5.8S, 5S, 12S and 16S rRNAs.

12. A method of amplifying nrRNA fragments, wherein the method comprises:
(i) separating nrRNA fragments from rRNA and rRNA fragments according to the method of claim 1, and
(ii) amplifying the nrRNA fragments.

13. A method of analyzing nrRNA expression, wherein the method comprises:
(i) separating nrRNA fragments from rRNA and rRNA fragments according to the method of claim 1, and
(ii) analyzing nrRNA expression.

14. A method of amplifying a cDNA complementary to an nrRNA fragment, wherein the method comprises:
(i) separating an nrRNA fragment from rRNA and rRNA fragments according to the method of claim 1, wherein the nrRNA fragment comprises a 5′ end;
(ii) ligating a first oligonucleotide to the 5′ end of the nrRNA fragment;
(iii) hybridizing to the nrRNA fragment a second oligonucleotide;
(iv) extending a DNA strand from the second oligonucleotide to generate a first cDNA;
(v) amplifying the first cDNA,
thereby amplifying a cDNA complementary to an nrRNA fragment.

15. A method of amplifying an nrRNA fragment, wherein the method comprises:
(i) separating an nrRNA fragment from rRNA and rRNA fragments according to the method of claim 1, wherein the nrRNA fragment comprises a 5′ end and a 3′ end;
(ii) ligating a first oligonucleotide to the 5′ end of the nrRNA fragment;
(iii) hybridizing to the nrRNA fragment a second oligonucleotide;
(iv) extending a DNA strand from the second oligonucleotide to generate a first cDNA having a 5′ end and a 3′ end;
(v) degrading the nrRNA fragment and, optionally, removing any free oligonucleotides and/or free RNA fragments;
(vi) synthesizing a complementary strand to the first cDNA by
(a) contacting the first cDNA with a third oligonucleotide such that the third oligonucleotide hybridizes to the 3′ end of the first cDNA, wherein the third oligonucleotide hybridizes to at least a portion of the first oligonucleotide and wherein the third oligonucleotide optionally contains an RNA polymerase promoter sequence, and
(b) extending the complementary strand from the third oligonucleotide, whereupon a double-stranded DNA copy of the nrRNA fragment is generated;
(vii) optionally purifying the double-stranded DNA copy; and
(viii) transcribing RNA from the double-stranded DNA copy,
thereby amplifying the nrRNA fragment.

16. The method of claim 15, wherein the second oligonucleotide comprises a unique sequence, a random sequence, and, optionally, an RNA polymerase promoter sequences, wherein the random sequence is of sufficient length to hybridize to the nrRNA.

17. A method of determining the level of nrRNA in a sample, wherein the method comprises:
(i) separating nrRNA fragments from rRNA and rRNA fragments according to the method of claim 1;
(ii) labeling the separated nrRNA fragments with a detectable label;
(iii) providing an array comprising a collection of fixed DNAs that hybridize to a plurality of target sequences located within an expression sequence of a gene of interest, wherein the fixed DNAs comprise from about 15 nucleotides to about 750 nucleotides, wherein at least one target sequence is interspersed approximately every 500 base pairs of the expression sequence;
(iv) applying the labeled nrRNA fragments to the array under hybridization conditions;
(v) optionally removing unbound and nonspecifically bound nrRNA fragments from the array;
(vi) detecting the labeled nrRNA fragments that have specifically annealed to the fixed DNAs in the array; and
(vii) quantitating the level of labeled nrRNA fragments detected in step (vi) to thereby determine the level of nrRNA in the sample.

18. The method of claim 17, wherein the method further comprises (viii) correlating the level of nrRNA to a level of expression of the gene of interest.

19. A kit for separating non-ribosomal transcribed RNA (nrRNA) fragments from a sample comprising ribosomal RNA (rRNA) and rRNA fragments, wherein the kit comprises a plurality of probes that hybridize to (a) different rRNA target sequences of at least 50% of contiguous regions of about 100 base pairs of the rRNA, which contiguous regions comprise the rRNA targeting sequences and (b) rRNA fragments comprising the rRNA targeting sequences hybridized to by the probes in the contiguous regions of the rRNA.

20. The kit of claim 19, wherein the probes hybridize to rRNA targeting sequences of at least 60% of the contiguous regions.

21. The kit of claim 20, wherein the probes hybridize to rRNA targeting sequences of at least 70% of the contiguous regions.

22. The kit of claim 21, wherein the probes hybridize to rRNA targeting sequences of at least 80% of the contiguous regions.

23. The kit of claim 22, wherein the probes hybridize to rRNA targeting sequences of at least 90% of the contiguous regions.

24. The kit of claim 23, wherein the probes hybridize to rRNA targeting sequences of at least 95% of the contiguous regions.

25. The kit of claim 24, wherein the probes hybridize to rRNA targeting sequences of all of the contiguous regions.

26. The kit of claim 19, wherein the rRNA and rRNA fragments comprise eukaryotic rRNA and eukaryotic rRNA fragments.

27. The kit of claim 19, wherein the rRNA and rRNA fragments comprise 28S, 18S, 5.8S, 5S, 16S, and 12S rRNAs or fragments thereof.

28. The kit of claim 19 further comprising instructions.

29. A system for separating non-ribosomal transcribed RNA (nrRNA) fragments from a sample comprising ribosomal RNA (rRNA) and rRNA fragments, the system comprising:
(i) a device adapted for the separation of nucleic acids by hybridization; and
(ii) a plurality of probes that hybridize to (a) different rRNA target sequences of at least 50% of contiguous regions of about 100 base pairs of the rRNA, which contiguous regions comprise the rRNA targeting sequences, and (b) rRNA fragments comprising the rRNA targeting sequences hybridized to by the probes in the contiguous regions of the rRNA, wherein the plurality of probes is positioned within the device to separate the rRNA and rRNA fragments from nrRNA.

30. The system of claim 29, wherein the probes hybridize to rRNA targeting sequences of at least 60% of the contiguous regions.

31. The system of claim 30, wherein the probes hybridize to rRNA targeting sequences of at least 70% of the contiguous regions.

32. The system of claim 31, wherein the probes hybridize to rRNA targeting sequences of at least 80% of the contiguous regions.

33. The system of claim 32, wherein the probes hybridize to rRNA targeting sequences of at least 90% of the contiguous regions.

34. The system of claim 33, wherein the probes hybridize to rRNA targeting sequences of at least 95% of the contiguous regions.

35. The system of claim 34, wherein the probes hybridize to rRNA targeting sequences of all of the contiguous regions.

36. The system of claim 29, wherein the rRNA and rRNA fragments comprise 28S, 18S, 5.8S, 5S, 16S, and 12S rRNAs or fragments thereof.
